(19) United States
(12) Patent Application Publication
(10) Pub. No.: US 2024/0426835 A1
(43) Pub. Date: Dec. 26, 2024

(54) Random Heteropolymer Excipients For High Protein Concentration Formulations

(71) Applicant: Genentech, Inc., South San Francisco, CA (US)

(72) Inventors: Ann Marie Woys, South San Francisco, CA (US); Kathryn Margaret Mansfield Messina, South San Francisco, CA (US)

(73) Assignee: Genentech, Inc., South San Francisco, CA (US)

(21) Appl. No.: 18/825,647

(22) Filed: Sep. 5, 2024

Related U.S. Application Data
(63) Continuation of application No. PCT/US23/63767, filed on Mar. 6, 2023.
(60) Provisional application No. 63/317,648, filed on Mar. 8, 2022.

(51) Int. Cl. G01N 33/68; C08F 220/14; C08F 220/18; C08F 220/28; C08F 220/34; C08F 220/60

(52) U.S. Cl. CPC G01N 33/6845; C08F 220/14; C08F 220/1811; C08F 220/286; C08F 220/34; C08F 220/60

(57) ABSTRACT

This application relates to random polymer libraries and specific polymers that can be used, for example, in stabilizing high concentration protein compositions, such as high concentration antibody compositions.

### CROSS REFERENCE TO RELATED APPLICATIONS

This application is a continuation of International Patent Application No. PCT/US2023/063767, filed Mar. 6, 2023, which claims priority to U.S. Application No. 63/317,648, filed Mar. 8, 2022, the entire contents of which are incorporated by reference herein for all purposes.

### FIELD

This application relates to random polymer libraries and specific polymers that can be used, for example, in stabilizing high concentration protein compositions, such as high concentration antibody compositions.

### BACKGROUND

Many protein compositions, such as pharmaceutical formulations, contain relatively low concentrations of the protein active ingredients, which can limit the utility of those compositions, for example, by limiting the way that they can be administered. For instance, many protein therapeutics must be administered intravenously in large volumes of solution as they cannot be concentrated sufficiently for other modes of administration that require lower volumes for administration, such as subcutaneous injection, intravitreal injection, intraocular administration, intranasal administration, inhalation, topical administration, and other methods. Thus, there is a continuous need for methods to stabilize protein-containing compositions at high concentration that allow for adequate shelf-life of the compositions and that are also both operable and safe for therapeutic use.

Some high concentration protein formulations, even though relatively stable, can have high viscosity, for example, which can limit their manufacturability as well as the type of administration. Certain small molecule excipients such as arginine and other amino acids may lower viscosity on a case-by-case basis, depending upon the protein in question, but are not effective for all proteins. In addition, such excipients may not be safe for all modes of therapeutic administration, and may in some cases be insufficient to stabilize a protein during storage or upon administration. Small molecule excipients, such as amino acids, may also diffuse away from the protein species upon administration or dilution of a protein formulation, which might lower the stability of the protein. Thus, there is a need for new methods to assist in reducing viscosity of high concentration protein formulations. In addition, there is a need to identify new excipients that may help limit precipitation of protein formulations and/or that do not diffuse away from proteins as quickly as small molecule excipients upon dilution or in vivo administration.

### SUMMARY

The present disclosure describes, inter alia, polymers having higher molecular weights than traditional small molecule protein formulation stabilizers such as amino acids and sugars and sugar alcohols, for example, with greater than 10-fold higher molecular weights than such small molecule excipients. Such polymeric excipients, for example, may be used to stabilize high concentration protein formulations, such as by limiting viscosity and/or inhibiting precipitation. The disclosure includes multiple embodiments, including, but not limited to, the following embodiments.

Embodiment 1 is a random polymer library comprising a mixture of polymers, wherein the library comprises polymers comprising at least three monomers chosen from: (a) methyl methacrylate (MMA), (b) oligo(ethylene glycol) methyl ether methacrylate (OEGMA), (c) isobutyl methacrylate (IBMA) or butyl methacrylate (BMA), and (d) a compound of the Formula I,

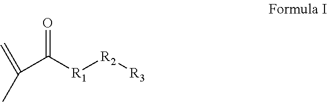

wherein R₁ is O or NH, R₂ is methyl, ethyl, propyl, or butyl, and R₃ is NH₂ or N(CH₃)₂ or a guanidinium group, wherein the library comprises polymers of from 3 to 20 kDa.

Embodiment 2 is the random polymer library of embodiment 1, wherein the library comprises polymers comprising a monomer of the Formula I, wherein the monomer is dimethyl amino methacrylate (DMAEMA), 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide.

Embodiment 3 is the random polymer library of embodiment 1 or 2, wherein at least 50%, at least 60%, at least 70%, at least 80%, or at least 90% of the polymers in the library comprise at least three monomers, or wherein the random polymer library consists essentially of polymers comprising at least three monomers.

Embodiment 4 is the random polymer library of embodiment 1 or 2, wherein at least 50%, at least 60%, at least 70%, at least 80%, or at least 90% of the polymers in the library comprise at least four monomers, or wherein the random polymer library consists essentially of polymers comprising at least four monomers.

Embodiment 5 is a random polymer library comprising a mixture of polymers, each polymer comprising a mixture of at least three of monomers: methyl methacrylate (MMA), oligo(ethylene glycol) methyl ether methacrylate (OEGMA), isobutyl methacrylate (IBMA), and dimethyl amino methacrylate (DMAEMA), wherein the library comprises polymers of from 3 to 20 kDa.

Embodiment 6 is a random polymer library comprising a mixture of polymers, each polymer comprising a mixture of at least three of monomers: methyl methacrylate (MMA), oligo(ethylene glycol) methyl ether methacrylate (OEGMA), isobutyl methacrylate (IBMA), and N-(3-methacrylamidopropyl) guanidinium chloride (ArgMAm), wherein the library comprises polymers of from 3 to 20 kDa.

Embodiment 7 is the random polymer library of any one of embodiments 1-6, wherein the library comprises polymers of from 3 to 15 kDa, from 5 to 20 kDa, from 5 to 15 kDa, from 5 to 10 kDa, from 10 to 20 kDa, from 7 to 10 kDa, or from 10 to 15 kDa.

Embodiment 8 is the random polymer library of any one of embodiments 1-7, wherein the library comprises OEGMA with a molecular weight of from 300 to 1500 g/mol.

Embodiment 9 is the random polymer library of embodiment 8, wherein the OEGMA has a molecular weight of 300, 500, 750, 950, 1000, 1200, or 1500 g/mol.

Embodiment 10 is the random polymer library of embodiment 8, wherein the OEGMA has a molecular weight of 500 g/mol (i.e., is OEGMA 500).

Embodiment 11 is the random polymer library of any one of embodiments 1˜4 or 7-10, wherein library comprises polymers comprising three monomers.

Embodiment 12 is the random polymer library of embodiment 11, wherein at least 50%, at least 60%, at least 70%, at least 80%, or at least 90% of the polymers in the library comprise three monomers, or wherein the random polymer library consists essentially of polymers comprising three monomers.

Embodiment 13 is the random polymer library of embodiment 11 or 12, wherein the three monomers are MMA, OEGMA, and either IBMA or BMA.

Embodiment 14 is the random polymer library of embodiment 13, wherein the three monomers are MMA, OEGMA, and IBMA.

Embodiment 15 is the random polymer library of embodiment 13, wherein the MMA, OEGMA, and IBMA or BMA are present in a ratio of: (a) 5:3:2, or (b) 3:5:2, or (c) 5:4:1.

Embodiment 16 is the random polymer library of any one of embodiments 1-10, wherein the library comprises polymers comprising four monomers.

Embodiment 17 is the random polymer library of embodiment 16, wherein at least 50%, at least 60%, at least 70%, at least 80%, or at least 90% of the polymers in the library comprise three monomers, or wherein the random polymer library consists essentially of polymers comprising three monomers.

Embodiment 18 is the random polymer library of embodiment 16 or 17, wherein the four monomers are MMA, OEGMA, IBMA, and DMAEMA.

Embodiment 19 is the random polymer library of embodiment 18, wherein the polymer library comprises polymers comprising 20-50% MMA, 20-50% OEGMA, 5-25% IBMA, and 5-25% DMAEMA by total weight of polymer.

Embodiment 20 is the random polymer library of embodiment 18, wherein the polymer library comprises polymers comprising MMA, OEGMA, IBMA, and DMAEMA in a molar ratio of: (a) 5:2.5:2:0.5, (b) 2:5:2:1, (c) 5:2.5:1:1.5, (d) 4:4:1:1; (e) 3:4:1:2; (f) 2:4:1:3; and/or (g) 2:3:1:4.

Embodiment 21 is the random polymer library of embodiment 18, wherein the MMA, OEGMA, IBMA, and DMAEMA are in a molar ratio of 5:2.5:2:0.5, wherein the OEGMA has a molecular weight of 500 g/mol (i.e., is OEGMA 500), wherein the library comprises polymers with a molecular weights of 3-15 kDa.

Embodiment 22 is the random polymer library of embodiment 16 or 17, wherein the four monomers are MMA, OEGMA, IBMA, and ArgMAm.

Embodiment 23 is the random polymer library of embodiment 22, wherein the polymer library comprises polymers comprising 20-50% MMA, 20-50% OEGMA, 5-25% IBMA, and 5-25% ArgMAm by total weight of polymer.

Embodiment 24 is the random polymer library of embodiment 22, wherein the polymer library comprises polymers comprising MMA, OEGMA, IBMA, and ArgMAm in a molar ratio of: (a) 5:2.5:2:0.5, (b) 2:5:2:1, (c) 5:2:1:2, (d) 4:4:1:1; (c) 3:4:1:2; (f) 2:4:1:3; and/or (g) 2:3:1:4.

Embodiment 25 is the random polymer library of embodiment 22, wherein the MMA, OEGMA, IBMA, and ArgMAm are in a molar ratio of 5:2:1:2 or 4:4:1:1, wherein the OEGMA has a molecular weight of 500 g/mol (i.e., is OEGMA 500), wherein the library comprises polymers with a molecular weights of 3-15 kDa.

Embodiment 26 is a random polymer library comprising a mixture of polymers, wherein the library comprises polymers comprising at least two monomers chosen from oligo(ethylene glycol) methyl ether methacrylate (OEGMA) and either isobutyl methacrylate (IBMA) or butyl methacrylate (BMA), and/or at least two monomers chosen from oligo(ethylene glycol) methyl ether methacrylate (OEGMA) and N-(3-methacrylamidopropyl) guanidinium chloride (ArgMAm), optionally wherein the OEGMA has a molecular weight of 500 g/mol (i.e., is OEGMA 500), and optionally wherein the library comprises polymers with a molecular weights of 3-15 kDa.

Embodiment 27 is a random polymer comprising a mixture of at least three monomers chosen from: (a) methyl methacrylate (MMA), (b) oligo(ethylene glycol) methyl ether methacrylate (OEGMA), (c) isobutyl methacrylate (IBMA), or (d) butyl methacrylate (BMA), and a compound of the Formula I,

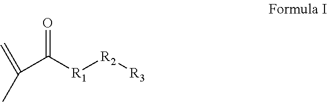

wherein R₁ is O or NH, R₂ is methyl, ethyl, propyl, or butyl, and R₃ is NH₂ or N(CH₃)₂ or a guanidinium group, wherein the polymer is from 3 to 20 kDa.

Embodiment 28 is the random polymer of embodiment 27, wherein the polymer comprises a monomer of the Formula I,

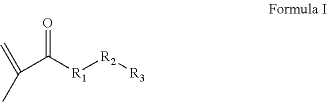

wherein the monomer is dimethyl amino methacrylate (DMAEMA), 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide.

Embodiment 29 is a random polymer comprising a mixture of at least three of monomers: methyl methacrylate (MMA), oligo(ethylene glycol) methyl ether methacrylate (OEGMA), isobutyl methacrylate (IBMA), and dimethyl amino methacrylate (DMAEMA), wherein the polymer is from 3 to 20 kDa.

Embodiment 30 is a random polymer comprising a mixture of at least three of monomers: methyl methacrylate (MMA), oligo(ethylene glycol) methyl ether methacrylate (OEGMA), isobutyl methacrylate (IBMA), and N-(3-methacrylamidopropyl) guanidinium chloride (ArgMAm), wherein the polymer is from 3 to 20 kDa.

Embodiment 31 is the random polymer of any one of embodiments 27-30, wherein the polymer is from 3 to 15 kDa, from 5 to 20 kDa, from 5 to 15 kDa, from 5 to 10 kDa, from 10 to 20 kDa, from 7 to 10 kDa, or from 10 to 15 kDa.

Embodiment 32 is the random polymer of any one of embodiments 27-31, wherein the polymer comprises OEGMA with a molecular weight of from 300 to 1500 g/mol.

Embodiment 33 is the random polymer of embodiment 32, wherein the OEGMA has a molecular weight of 300, 500, 750, 950, 1000, 1200, or 1500 g/mol.

Embodiment 34 is the random polymer of embodiment 33, wherein the OEGMA has a molecular weight of 500 g/mol.

Embodiment 35 is the random polymer of any one of embodiments 27-28 or 31-34, wherein the polymer comprises three monomers.

Embodiment 36 is the random polymer of embodiment 35, wherein the three monomers are MMA, OEGMA, and either IBMA or BMA.

Embodiment 37 is the random polymer of embodiment 36, wherein the three monomers are MMA, OEGMA, and IBMA.

Embodiment 38 is the random polymer of embodiment 37, wherein the MMA, OEGMA, and IBMA or BMA are present in a ratio of: (a) 5:3:2, or (b) 3:5:2, or (c) 5:4:1.

Embodiment 39 is the random polymer of any one of embodiments 27-34, wherein the polymer comprises four monomers.

Embodiment 40 is the random polymer of embodiment 39, wherein the four monomers are MMA, OEGMA, IBMA, and DMAEMA.

Embodiment 41 is the random polymer of embodiment 40, wherein the polymer comprises 20-50% MMA, 20-50% OEGMA, 5-25% IBMA, and 5-25% DMAEMA by total weight of polymer.

Embodiment 42 is the random polymer of embodiment 40, wherein the polymer comprises MMA, OEGMA, IBMA, and DMAEMA in a molar ratio of: (a) 5:2.5:2:0.5, (b) 2:5:2:1, (c) 5:2.5:1:1.5, (d) 4:4:1:1; (c) 3:4:1:2; (f) 2:4:1:3; or (g) 2:3:1:4.

Embodiment 43 is the random polymer of embodiment 40, wherein the MMA, OEGMA, IBMA, and DMAEMA are in a molar ratio of 5:2.5:2:0.5, wherein the OEGMA is OEGMA 500, wherein the polymer has a molecular weight of from 3 to 15 kDa.

Embodiment 44 is the random polymer of embodiment 39, wherein the four monomers are MMA, OEGMA, IBMA, and ArgMAm.

Embodiment 45 is the random polymer of embodiment 44, wherein the polymer comprises 20-50% MMA, 20-50% OEGMA, 5-25% IBMA, and 5-25% ArgMAm by total weight of polymer.

Embodiment 46 is the random polymer of embodiment 44, wherein the polymer comprises MMA, OEGMA, IBMA, and ArgMAm in a molar ratio of: (a) 5:2.5:2:0.5, (b) 2:5:2:1, (c) 5:2.5:1:1.5, (d) 4:4:1:1; (c) 3:4:1:2; (f) 2:4:1:3; or (g) 2:3:1:4.

Embodiment 47 is the random polymer of embodiment 44, wherein the MMA, OEGMA, IBMA, and ArgMAm are in a molar ratio of 5:2:1:2 or 4:4:1:1, wherein the OEGMA is OEGMA 500, wherein the polymer has a molecular weight of from 3 to 15 kDa.

Embodiment 48 is a random polymer comprising mixture of at least two monomers chosen from oligo(ethylene glycol) methyl ether methacrylate (OEGMA) and either isobutyl methacrylate (IBMA) or butyl methacrylate (BMA), and/or at least two monomers chosen from oligo(ethylene glycol) methyl ether methacrylate (OEGMA) and N-(3-methacrylamidopropyl) guanidinium chloride (ArgMAm), optionally wherein the OEGMA has a molecular weight of 500 g/mol (i.e., is OEGMA 500), and optionally wherein the polymer has a molecular weight of from 3 to 15 kDa.

Embodiment 49 is the random polymer of any one of embodiments 27-48, wherein the polymer has a molecular weight of from 7 to 10 kDa.

Embodiment 50 is a composition comprising a random polymer according to any one of embodiments 27-49 and at least one protein.

Embodiment 51 is the composition of embodiment 50, wherein the polymer is at a concentration of from 0.01 to 1% w/v, such as 0.01 to 0.1% w/v.

Embodiment 52 is the composition of embodiment 50 or 51, wherein the protein is an antibody.

Embodiment 53 is the composition of embodiment 52, wherein the antibody is an IgG antibody, such as a full-length IgG, a bi-specific IgG, or wherein the antibody is an antigen binding fragment, such as a Fab, Fab′, (Fab′)2, Fv, or scFv.

Embodiment 54 is the composition of any one of embodiments 50-53, wherein the composition further comprises at least one buffer, such as histidine, phosphate, or citrate.

Embodiment 55 is the composition of any one of embodiments 50-54, wherein the composition further comprises at least one surfactant, such as a polysorbate, for example polysorbate 20 or polysorbate 80.

Embodiment 56 is the composition of any one of embodiments 50-55, wherein the antibody is at a concentration of 10 mg/mL to 150 mg/mL, such as 10-100 mg/mL, 20-100 mg/mL, 20-80 mg/mL, 10-50 mg/mL, 10-25 mg/mL, 25-50 mg/mL, 50-80 mg/mL, 50-100 mg/mL, or 70-100 mg/mL.

Embodiment 57 is a method of reducing the viscosity and/or inhibiting precipitation of a protein-containing composition, comprising adding a random polymer of any one of embodiments 27-49 to the composition, optionally wherein the composition comprises the protein and/or excipients of any one of embodiments 50-56.

Embodiment 58 is a method of preparing a random polymer library according to any one of embodiments 1-26 or a random polymer according to any one of embodiments 27-49, wherein the method comprises reverse addition/fragmentation chain transfer (RAFT), free radical polymerization (FRP), or atom transfer radical polymerization (ATRP).

Embodiment 59 is a method of preparing a random polymer library according to any one of embodiments 1-26 or a random polymer according to any one of embodiments 27-49, the method comprising: exposing the monomers to LED light and/or heat in the presence of a zinc tetraphenyl porphyrin catalyst (ZnTPP) and a chain transfer agent.

Embodiment 60 is the method of embodiment 59, wherein the chain transfer agent is 2-cyano-2-propyl benzodithioate, 2-cyano-2-propyldodecyl trithiocarbonate, 4-((((2-carboxyethyl)thio) carbonothioyl)thio)-4-cyanopentanoic acid, 4-cyano-4-(phenylcarbonothioylthio) pentanoic acid, or 4-cyano-4-[(dodecylsulfanylthiocarbonyl) sulfanyl] pentanoic acid (CDTPA).

Embodiment 61 is the method of embodiment 59 or 60, wherein the monomers are exposed to LED light in the presence of the ZnTPP and chain transfer agent for 3 to 18 hours.

Embodiment 62 is the method of any one of embodiments 59-61, wherein polymerization is ended by removing the LED light.

Embodiment 63 is the method of any one of embodiments 58-62, further comprising performing at least one filtration, buffer exchange, or dialysis step to isolate the polymers.

Embodiment is a kit comprising the random polymer library of any one of embodiments 1-26, for use in testing polymers comprised within the library for their effect on precipitation, viscosity, and/or turbidity of a solution comprising a protein, the kit optionally further comprising instructions for use.

Embodiment 65 is the kit of embodiment 64, wherein the library comprises at least 10, at least 20, at least 50, or at least 80 different individual random polymers.

Embodiment 66 is a method of identifying a random polymer excipient for a protein solution, comprising exposing the protein solution to a random polymer library of any one of embodiments 1-26 or the kit of embodiment 64 or 65 and determining one or more of the viscosity, turbidity, or precipitation of the solution.

It is to be understood that both the foregoing general description and the following detailed description are exemplary and explanatory only and are not restrictive of the claims. References cited herein are incorporated herein by reference.

### BRIEF DESCRIPTION OF THE DRAWINGS

FIG. 1 shows an exemplary method of evaluating a polymer library for each polymer's effect on monoclonal antibody (mAb, such as mAb G) precipitation.

FIGS. 2A-B show turbidity measurements (absorbance at 600 nm) of mAb G (80 mg/mL) after incubation overnight in 15 mM phosphate buffer pH 7.4 with 100 mM NaCl (FIG. 2A) or with 150 mM NaCl (FIG. 2B) with selected polymer excipients at 1.6, 6.6, and 33 μM polymer (˜0.0014, 0.006, 0.03% polymer).

FIG. 3 shows precipitation kinetics of mAb G (80 mg/mL) in 15 mM phosphate buffer pH 7.4+150 mM NaCl with polymer excipients. Turbidity was measured using absorbance at 600 nM.

FIG. 4 shows precipitation kinetics of mAb G (80 mg/mL) in 15 mM phosphate buffer pH 7.4+150 mM NaCl with polysorbate 20 (PS20). Turbidity was measured using absorbance at 600 nM.

FIGS. 5A-B show changes in viscosity of mAb G solutions (194 mg/mL in 30 mM histidine chloride (HisCl) pH 5.5) with excipients Poly1D (“Poly1”), polysorbate 20 (PS20), and Poly1D in combination with PS20, at different excipient concentrations. FIG. 5A shows a graph of change in viscosity with increasing concentrations of either Poly1D or PS20. FIG. 5B shows a bar graph of changes in viscosity at particular concentrations of Poly1D, PS20, or a combination of the two.

FIG. 6 shows viscosity dependence of mAb G (194 mg/mL in HisCl pH 5.5) on shear rate, which is indicative of surface-active excipients.

FIGS. 7A-B show processing of samples for the turbidity/precipitation assay. FIG. 7A shows mAb G (80 mg/mL) after precipitation in 15 mM phosphate buffer pH 7.4+150 mM NaCl (A) from high throughput screen of mAb G controls (left, solid line box), with increasing Poly1E (middle, dashed line box), and with increasing PS20 (right, dotted line box). FIG. 7B shows mAb G with with PS20 (left) or Poly1E (right), after centrifugation.

FIG. 8 shows microscale thermophoresis (MST) measurements, which indicate weak binding between Poly1F (15.5 μM) with mAbs A-D (2×10⁻⁷ to 1×10₋₄ M) in HisCl pH 5.5.

FIG. 9 shows the average change in viscosity of 8 mAbs when incubated with a polymer library comprising increasing percent amounts of DMAEMA.

FIG. 10 shows the average change in viscosity of 8 mAbs when incubated with a polymer library comprising increasing percent amounts of IBMA in a polymer.

FIG. 11 shows the average change in viscosity of 8 mAbs when incubated with a polymer library comprising increasing percent amounts of OEGMA.

FIG. 12 shows the average change in viscosity of 8 mAbs when incubated with Poly1, Poly11, Poly3, Poly5, Poly7, or Poly9, each with a molecular weight of 10 kDa.

FIGS. 13A-B show HPLC traces of (13A) crude and (13B) purified Poly1H which confirm elimination of DMSO (retention time approximately 7 minutes).

FIGS. 14A-B show exemplary absorbance measurements for precipitation of exemplary polymers at 320 nm (14A) and 600 nm (14B).

FIG. 15 shows absorbance measurements for turbidity of exemplary polymers over time at 600 nm.

FIGS. 16A-D show absorbance measurements for turbidity of exemplary polymers over time at 600 nm.

FIG. 17 shows absorbance maximum (Aₘₐₓ) for exemplary polymers. Dark grey indicates little or no improvement, light grey indicates moderate improvement, and white indicates large improvement in precipitation kinetics attributes compared to control. Bold text indicates polymers with no dark grey classification and at least two white classifications.

FIGS. 18A-B show precipitation of mAB-G with and without exemplary polymers in 15 mM phosphate buffer pH 7.4 with 150 mM NaCl at 37° C.

FIGS. 19A-H show precipitation assessment with low-throughput physiologically relevant models. Solubility of mAb-G in phosphate buffer pH 7.4+150 mM NaCl at 37° C. in Eppondorf tubes (n=3) were assessed by (19A) the soluble concentration mAb-G and (19B) the precipitate weight of mAb-G after drying following separation of solids by centrifugation; **p<0.005 between Poly1 and no additive. Evaluation of mAB-G precipitation (representative of 3; 19C-E) and pharmacokinetics (PK; n=3; 19F) in the SCISSOR system. FIG. 19G shows Poly1 release from the SCISSOR cartridge (n=3). FIG. 19H shows DLS of Poly1 micelles in 20 mM HisCl pH 5.5

FIGS. 20A-B show SCISSOR images (20A) and transmission data (20B) from 0.2 mL injection of 50 mg/mL of mAb-G.

FIGS. 21A-D show biophysical characterization of mAb-G-Poly1 interactions using microscale thermophoresis (MST; 21A), isothermal titration calorimetry (ITC; representative of 3; 21B), and intrinsic fluorescence quenching (n=3; 21C) and corresponding Stern-Volmer plot (21D).

FIG. 22 shows cytotoxicity of Poly1 at several concentrations in Jurkat cells using trypan blue to assess cell membrane integrity (n=4).

FIG. 23 shows ITC curve of 1 mM mAb-G with 100 μM Poly1.

FIG. 24 shows 1H-NMR of ArgMAm in DMSO-d6.

FIG. 25 shows 1H-NMR of Poly1 in DMSO-d6.

FIG. 26 shows GPC trace of Poly1.

FIG. 27 shows dynamic light scattering (DLS) of Poly1 micelles in 30 mM HisCl pH 5.5.

FIG. 28A-D show pendant drop tensiometer ST measurements at 0.1% (28A), 0.02% (28B), and 0.002% (28C) concentrations of Poly1. FIG. 28D shows surface tension measurements in mN/m for 0.1% (28A), 0.02% (28B), and 0.002% (28C) concentrations of Poly1.

### DETAILED DESCRIPTION OF CERTAIN EMBODIMENTS

### I. Definitions

The present invention may be understood more readily by reference to the following detailed description of specific embodiments and the Examples included below.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Measured values are understood to be approximate, taking into account significant digits and the error associated with the measurement.

As used herein, percentages (“%”) are weight to volume (“w/v”) percentages unless specified otherwise.

The present disclosure relates to various protein-containing formulations. Such “formulations” may also be interchangeably called “compositions” or “preparations” or “solutions” herein.

“Polypeptide” or “protein” means a sequence of amino acids for which the chain length is sufficient to produce a tertiary structure. Thus, proteins herein are distinguished from “peptides,” which are short amino acid-based molecules that generally do not have any tertiary structure. Typically, a protein for use herein will have a minimum molecular weight of at least about 5-20 kD, alternatively at least about 15-20 kD, preferably at least about 20 kD. Polypeptides or proteins herein include, for example, antibodies.

The term “antibody” as used herein includes polyclonal antibodies, monoclonal antibodies (including full length antibodies which have an immunoglobulin Fc region), antibody compositions with polyepitopic specificity, bispecific and multispecific antibodies (including diabodies, one-armed antibodies, and single-chain molecules), as well as antigen-binding fragments (e.g., Fab, F(ab′)₂, scFv, and Fv). Antibodies herein comprise a set of complementary depending regions (CDRs) located in heavy (H) and light (L) chain variable domains that collectively recognize a particular antigen. Antibodies herein comprise at least the portions of the heavy and light chain variable domain amino acid sequences sufficient to include the set of CDRs for antigen recognition. In some embodiments, antibodies comprise full length heavy and light chain variable domains. In some embodiments, antibodies further comprise heavy and/or light chain constant regions, which may or may not be full length.

The term “immunoglobulin” (Ig) is used interchangeably with “antibody” herein.

The term, “stabilizing agent” or “stabilizer” as used herein is a chemical or compound that is added to a formulation to maintain it in a stable or unchanging state. In some cases, a stabilizer may be added to help prevent precipitation, aggregation, oxidation, color changes, or the like. Depending on the circumstances, stabilizers can include small molecules such as amino acids, sugars, and sugar alcohols, as well as certain proteins (e.g., albumin), surfactants, and polymers.

“Surfactants” are molecules with well-defined polar and non-polar regions that allow them to aggregate in solution to form micelles. Depending on the nature of the polar area, surfactants can be non-ionic, anionic, cationic, and zwitterionic.

A “polymer” as used herein refers to a molecule comprising a linear or branched chain of repeating monomer elements.

A “random polymer” is a polymer in which the individual, different monomers making up the polymer chain are not in a specific, pre-determined order along the length of the chain. A “random polymer library” or a “mixture of random polymers” interchangeably refer to a mixture of random polymers, for example, of different lengths, different molecular weights, and/or different molar ratios of the individual monomers. In some embodiments, a random polymer library may comprise random polymers having the same set of monomers, and the same monomer ratios, but having different chain lengths or molecular weights. In other embodiments, the library may comprise random polymers having the same chain lengths or molecular weights and having the same set of monomers, but with different molar ratios of the individual monomers. In some embodiments, the library may comprise random polymers having different sets of monomers, but for example, the same molar ratios of the different monomers and/or the same chain length.

A “chain transfer agent” as used herein refers to a molecule added to a polymerization reaction that may act to control or slow the growth of the polymer chain during polymerization.

A “buffer” may be included in some protein formulations, and is a substance that helps to control the pH of the formulation. Examples of buffers used in therapeutic formulations include, for instance, phosphate, citrate, and histidine. Laboratory formulations may often include buffers such as Tris, HEPES, and the like.

The term “pharmaceutical formulation” or “therapeutic formulation” or “therapeutic preparation” refers to a preparation or composition comprising at least one active ingredient (e.g. a protein) and at least one additional component or excipient substance, and which is in such form as to permit the biological activity of the active ingredient to be effective in an animal subject, such as a mammal, and which is “suitable for therapeutic use” or “suitable for pharmaceutical use,” meaning that the formulation as a whole is not unacceptably toxic to a subject and does not contain components which are unacceptably toxic to a subject to which the formulation would be administered or which are at concentrations that would render them unacceptably toxic to a subject.

A “stable” formulation is one in which the protein therein essentially retains its physical and/or chemical stability upon storage and administration. Stability can be measured at a selected temperature for a selected time period. Various analytical techniques for measuring protein stability are available in the art and are reviewed, for example, in Peptide and Protein Drug Delivery, 247-301, Vincent Lec Ed., Marcel Dekker, Inc., New York, N. Y., Pubs. (1991) and Jones, A. Adv. Drug Delivery Rev. 10:29-90 (1993).

Increasing the “stability” of a protein-containing formulation may involve reducing (as compared to an untreated protein-containing formulation) or preventing the formation of precipitates, protein aggregates or degradation products, reducing oxidation and color changes, and the like.

The term “aggregate” or “aggregation” as used herein means to come together or collect in a mass or whole, e.g., as in the aggregation of protein molecules. Aggregates can be self-aggregating or aggregate due to other factors, e.g., presence of aggregating agents, precipitating agents, agitation, or other means and methods whereby proteins cause to come together. Aggregation may be observed visually, such as when a previously clear protein formulation in solution becomes cloudy or contains precipitates, or by methods such as size exclusion chromatography (SEC), which separates proteins in a formulation by size. Aggregates may include dimers, trimers, and multimers of the protein species. As used herein, “high molecular weight species” (HMWS) refers to aggregates of proteins that may, for example, be observed by size exclusion chromatography, and that represent at least dimers of the desired protein molecules, i.e., having at least twice the molecular weight of the desired protein species in a formulation. In the case of a protein species such as an antibody that, in its normal or desired form is already a multimer, e.g. a dimer or tetramer, a HMWS would represent at least a dimer of the normal, desired multimeric form of the protein.

“Isolated” when used to describe the various polypeptides and antibodies disclosed herein, means a polypeptide or antibody that has been identified, separated and/or recovered from a component of its production environment. Optionally, the isolated polypeptide is also free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes.

In some embodiments herein, pharmaceutical formulations “do not comprise” one or more types of excipients or ingredients such as a surfactant or an amino acid excipient or the like. The expression “does not comprise” in this context means that the excluded ingredients are not present beyond trace levels, for example, due to contamination or impurities found in other purposefully added ingredients.

The term “consisting essentially of” when referring to a mixture of ingredients of a formulation herein indicates that, while ingredients other than those expressly listed may be present, such ingredients are found only in trace amounts or in amounts otherwise low enough that certain fundamental characteristics of the formulation including protein concentration, such as its level of protein precipitation, turbidity, and viscosity are unchanged.

### II. Exemplary Random Polymer Libraries and Selected Polymers

Embodiments herein include random polymer libraries made from a plurality of random polymers. Embodiments here also include particular random polymers, such as those having a particular relative concentration of a specific set of monomers and a particular average chain length or molecular weight.

In some cases, the random polymers of the random polymer library comprise a mixture of at least three different monomers, such as acrylate and/or acrylamide monomers. In some cases, at least 50%, at least 60%, at least 70%, at least 80%, at least 90% or at least 95% of the polymers in the library have at least three monomers, for example, with the remaining polymers in the library having one to two monomers. In some cases, at least 50%, at least 60%, at least 70%, at least 80%, at least 90% or at least 95% of the polymers in the library have at least four monomers, for example, with the remaining polymers in the library having one to three monomers. In some cases, at least 50%, at least 60%, at least 70%, at least 80%, at least 90% or at least 95% of the polymers in the library have three monomers, for example, with the remaining polymers in the library having one or two or four, or more than four monomers or a mixture of these possibilities. In some cases, at least 50%, at least 60%, at least 70%, at least 80%, at least 90% or at least 95% of the polymers in the library have four monomers, for example, with the remaining polymers in the library having one, two, or three, or more than four monomers or a mixture of these possibilities.

In some cases, the polymers in the library consist essentially of at least two, at least three, or at least four monomers. In some embodiments, the random polymer library consists essentially of polymers with three or four monomers. In some cases, the library consists essentially of polymers with three monomers. In other cases, the library consists essentially of polymers with four monomers. In such cases, the polymers in the library are each intended to consist of a particular number of monomers, but, due to the complexities of chemical synthesis, the library may contain small amounts of polymers with a different number of monomers.

In some cases, a random polymer library comprises polymers with a mixture of at least three monomers chosen from methyl methacrylate (MMA), oligo(ethylene glycol) methyl ether methacrylate (OEGMA), isobutyl methacrylate (IBMA), butyl methacrylate (BMA), dimethyl amino methacrylate (DMAEMA), (4-hydroxyphenyl) methacrylamide, 2-hydroxyethyl methacrylate, [2-(methacryloyloxy)ethyl] dimethyl-(3-sulfopropyl) ammonium hydroxide, 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide. In some cases, a random polymer library comprises polymers with a mixture of at least three monomers chosen from oligo(ethylene glycol) methyl ether methacrylate (OEGMA) and any of the following additional monomers: methyl methacrylate (MMA), isobutyl methacrylate (IBMA), butyl methacrylate (BMA), dimethyl amino methacrylate (DMAEMA), (4-hydroxyphenyl) methacrylamide, 2-hydroxyethyl methacrylate, [2-(methacryloyloxy)ethyl] dimethyl-(3-sulfopropyl) ammonium hydroxide, 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide. In some cases, the library comprises polymers with only one of isobutyl methacrylate (IBMA) and butyl methacrylate (BMA), and not both. In some cases, only one of dimethyl amino methacrylate (DMAEMA), (4-hydroxyphenyl) methacrylamide, 2-hydroxyethyl methacrylate, [2-(methacryloyloxy)ethyl] dimethyl-(3-sulfopropyl) ammonium hydroxide, 2-aminoethyl methacrylate, arginine methacrylate, arginine methacrylamide, N-(3-aminopropyl) methacrylamide, N-(3-methacrylamidopropyl) guanidinium chloride (ArgMAm), and N-3-(dimethylamino) propyl methacrylamide is chosen.

In some embodiments herein, the random polymer library comprises polymers comprising three or more monomers chosen from: (a) methyl methacrylate (MMA), (b) oligo(ethylene glycol) methyl ether methacrylate (OEGMA), (c) either isobutyl methacrylate (IBMA) or butyl methacrylate (BMA). In some embodiments herein, the random polymer library comprises three or more monomers chosen from: (a) methyl methacrylate (MMA), (b) oligo(ethylene glycol) methyl ether methacrylate (OEGMA), (c) either isobutyl methacrylate (IBMA) or butyl methacrylate (BMA), and (d) at least one compound of the Formula I,

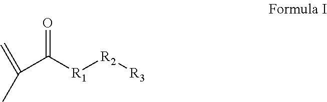

wherein R₁ is O or NH, R₂ is methyl, ethyl, propyl, or butyl, and R₃ is NH₂ or N(CH₃)₂ or OH, or wherein R₁ is O or NH and R₂ and R₃ together comprise hydroxyphenyl, such as 4-hydroxyphenyl, or wherein R₁ is O or NH and R₂ is an N,N-dimethylethylamine group and R₃ is a sulfopropyl group. Such a library comprises, for example, monomers from (a), (b), and (c); or alternatively (a), (b), and (d); or (b), (c), and (d); or (a), (c), and (d); or each one of (a)-(d), etc., and thereby, comprises at least three different monomers total. In some cases, a library could comprise a mixture of polymers, each one with a different set of monomers from among these options. In other cases, a library could contain polymers each with the same set of monomers, but in different relative concentrations and/or at different lengths or molecular weights as determined by the reaction conditions.

In some embodiments, the disclosure comprises a random polymer library comprising polymers comprising a mixture of at least three monomers chosen from: (a) methyl methacrylate (MMA), (b) oligo(ethylene glycol) methyl ether methacrylate (OEGMA), (c) isobutyl methacrylate (IBMA) or butyl methacrylate (BMA), and (d) a compound of the Formula I,

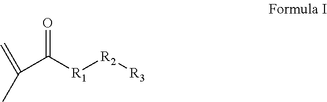

wherein R₁ is O or NH, R₂ is methyl, ethyl, propyl, or butyl, and R₃ is NH₂ or N(CH₃)₂ or a guanidinium group (e.g., H₂N—C(═NH₂⁺)—NH₂) wherein the library comprises polymers of from 3 to 20 kDa. As above, such a library comprises, for example, monomers from (a), (b), and (c); or alternatively (a), (b), and (d); or (b), (c), and (d); or (a), (c), and (d); or each of (a)-(d), etc., and thereby, comprises at least three different monomers total. And, as above, in some cases, a library could comprise a mixture of polymers, each one with a different set of monomers from among these options. In other cases, a library could contain polymers each with the same set of monomers, but in different relative concentrations and/or at different lengths or molecular weights as determined by the reaction conditions. In some cases, the library comprises polymers comprising a monomer of the Formula I, wherein the monomer is dimethyl amino methacrylate (DMAEMA), 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide. In some cases, the library comprises polymers comprising at least three of: methyl methacrylate (MMA), oligo(ethylene glycol) methyl ether methacrylate (OEGMA), isobutyl methacrylate (IBMA), and dimethyl amino methacrylate (DMAEMA), or comprises four of those monomers. In some cases, a random polymer library comprises at least one monomer of Formula I, chosen from dimethyl amino methacrylate (DMAEMA), 2-hydroxyethyl methacrylate, 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide.

In any of the above cases, the library sometimes comprises polymers with three monomers. In other cases, the library comprises polymers with four monomers. In yet other cases, five monomers are used.

In some cases, the library comprises polymers of three monomers, wherein the three monomers are MMA, OEGMA, and either IBMA or BMA. In some cases, the three monomers are MMA, OEGMA, and IBMA. In some cases, the library comprises polymers of four monomers: MMA, OEGMA, IBMA or BMA, and one of: DMAEMA, arginine methacrylate, arginine methacrylamide, N-(3-aminopropyl) methacrylamide, or N-(3-methacrylamidopropyl) guanidinium chloride (ArgMAm). In some cases the four monomers are MMA, OEGMA, IBMA or BMA, and DMAEMA or ArgMAm. In some cases the four monomers are MMA, OEGMA, IBMA or BMA, and DMAEMA. In some cases, the four monomers are MMA, OEGMA, IBMA or BMA, and ArgMAm. In some cases the four monomers are MMA, OEGMA, IBMA, and DMAEMA or ArgMAm. In some cases the four monomers are MMA, OEGMA, IBMA, and DMAEMA. In some cases the four monomers are MMA, OEGMA, IBMA, and ArgMAm. In some cases, polymers of the library comprise a mix of both IBMA and BMA as well as MMA and OEGMA. In some cases, polymers of the library comprise a mix of both IBMA and BMA. In some cases, polymers of the library comprise more than one compound of Formula I, such as both DMAEMA and ArgMAm.

In other embodiments, the random polymer library comprises polymers with two monomers. In some embodiments, the two monomers are BMA or IBMA and OEGMA. In some cases, the two monomers are IBMA and OEGMA. In some cases, the two monomers are BMA and OEGMA. In other cases, the two monomers are ArgMAm and OEGMA.

In any of the above cases, in some embodiments the library comprises polymers of from 3 to 20 kDa. In some embodiments, the library comprises polymers of from the library comprises polymers of from 3 to 15 kDa, from 5 to 20 kDa, from 5 to 15 kDa, from 5 to 10 kDa, from 5 to 8 kDa, from 10 to 20 kDa, from 7 to 10 kDa, from 8 to 10 kDa, from 10 to 12 kDa, from 12 to 15 kDa, or from 10 to 15 kDa. For example, a polymer library may be created having polymers of various lengths, and as a result, various molecular weights.

When OEGMA is present, in some embodiments, the library comprises OEGMA with a molecular weight of from 300 to 1500 g/mol. In some such embodiments, the OEGMA may have a molecular weight of 300, 500, 750, 950, 1000, 1200, or 1500 g/mol. In some cases, it has a molecular weight of 500 g/mol (i.e., OEGMA 500). In some cases, a random polymer library may also be created with the size of the OEGMA as a variable to be altered, i.e. such that the library comprises polymers with different sizes of OEGMA but otherwise, for example, the same set of monomers, optionally in the same relative concentrations and/or with the same approximate chain length.

In some embodiments, a random polymer library may be made comprising polymers with a range of molar ratios of the monomers, or comprising polymers with one particular set of molar ratios, but polymers that differ, for example in length, or in molecular weight of OEGMA (if OEGMA is present) or varying in another changeable parameter. In some embodiments, polymers of the library comprise 20-50% MMA, 20-50% OEGMA, and 5-25% IBMA by total weight of polymer. In some such cases, the polymers are made from monomers consisting essentially of DMAEMA, IBMA, and OEGMA, for example, at 20-50% MMA, 20-50% OEGMA, and 5-25% IBMA by total weight of polymer. In other cases, at least one additional monomer is also present, such as DMAEMA or ArgMAm. In some cases, the polymers may comprise 20-50% MMA, 20-50% OEGMA, 5-25% IBMA, and 5-25% DMAEMA. In some embodiments, polymers of the library comprise DMAEMA or ArgMAm, IBMA, OEGMA, and MMA, and optionally, are present as follows: MMA, OEGMA, IBMA, and DMAEMA or ArgMAm in a molar ratio of: (a) 5:2.5:2:0.5, (b) 2:5:2:1, (c) 5:2.5:1:1.5, (d) 4:4:1:1; (c) 3:4:1:2; (1) 2:4:1:3; (g) 2:3:1:4, or (h) 5:2:1:2. In some embodiments, the MMA, OEGMA, IBMA, and DMAEMA are in a molar ratio of 5:2.5:2:0.5, wherein the OEGMA has a molecular weight of 500 g/mol (i.e., is OEGMA 500), wherein the library comprises polymers with a molecular weights of 3-15 kDa. In some embodiments, a polymer library may be made from MMA, OEGMA, and IBMA in a molar ratio of 5:4:1 or of 5:3:2 or of 3:5:2. And such a polymer library may have a range of molecular weights of from 3 to 15 kDa. In some cases, the OEGMA may be OEGMA 500.

Embodiments herein include random polymers made from a mixture of at least three different monomers, such as acrylate and/or acrylamide monomers. In some cases, a random polymer comprises a mixture of at least three monomers chosen from methyl methacrylate (MMA), oligo(ethylene glycol) methyl ether methacrylate (OEGMA), isobutyl methacrylate (IBMA), butyl methacrylate (BMA), dimethyl amino methacrylate (DMAEMA), (4-hydroxyphenyl) methacrylamide, 2-hydroxyethyl methacrylate, [2-(methacryloyloxy)ethyl] dimethyl-(3-sulfopropyl) ammonium hydroxide, 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide. In some cases, a polymer comprises a mixture of at least three monomers chosen from oligo(ethylene glycol) methyl ether methacrylate (OEGMA) and any of the following additional monomers: methyl methacrylate (MMA), isobutyl methacrylate (IBMA), butyl methacrylate (BMA), dimethyl amino methacrylate (DMAEMA), (4-hydroxyphenyl) methacrylamide, 2-hydroxyethyl methacrylate, [2-(methacryloyloxy)ethyl] dimethyl-(3-sulfopropyl) ammonium hydroxide, 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide. In some cases, the polymer comprises only one of isobutyl methacrylate (IBMA) and butyl methacrylate (BMA), and not both.

In some embodiments herein, the random polymer comprises three or more monomers chosen from: (a) methyl methacrylate (MMA), (b) oligo(ethylene glycol) methyl ether methacrylate (OEGMA), (c) either isobutyl methacrylate (IBMA) or butyl methacrylate (BMA), and (d) at least one compound of the Formula I,

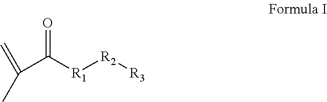

wherein R₁ is O or NH, R₂ is methyl, ethyl, propyl, or butyl, and R₃ is NH₂ or N(CH₃)₂ or OH, or wherein R₁ is O or NH and R₂ and R₃ together comprise hydroxyphenyl, such as 4-hydroxyphenyl, or wherein R₁ is O or NH and R₂ is an N,N-dimethylethylamine group and R₃ is a sulfopropyl group. Such a polymer comprises, for example, monomers from (a), (b), and (c); or alternatively (a), (b), and (d); or (b), (c), and (d); or (a), (c), and (d); or each of (a)-(d), etc., and thereby, comprises at least three different monomers total.

In some embodiments, the disclosure comprises a random polymer comprising a mixture of at least three monomers chosen from: (a) methyl methacrylate (MMA), (b) oligo(ethylene glycol) methyl ether methacrylate (OEGMA), (c) isobutyl methacrylate (IBMA) or butyl methacrylate (BMA), and (d) a compound of the Formula I,

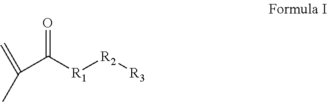

wherein R₁ is O or NH, R₂ is methyl, ethyl, propyl, or butyl, and R₃ is NH₂ or N(CH₃)₂ or a guanidinium group, wherein the library comprises polymers of from 3 to 20 kDa. As above, such a polymer comprises, for example, monomers from (a), (b), and (c); or alternatively (a), (b), and (d); or (b), (c), and (d); or (a), (c), and (d); or (a)-(d), etc., and thereby, comprises at least three different monomers total. In some cases, the polymer comprises a monomer of the Formula I, wherein the monomer is dimethyl amino methacrylate (DMAEMA), 2-aminoethyl methacrylate, arginine methacrylate, arginine methacrylamide, N-(3-aminopropyl) methacrylamide, or N-3-(dimethylamino) propyl methacrylamide. In some cases, the polymer comprises at least three of: methyl methacrylate (MMA), oligo(ethylene glycol) methyl ether methacrylate (OEGMA), isobutyl methacrylate (IBMA), dimethyl amino methacrylate (DMAEMA), and N-(3-methacrylamidopropyl) guanidinium chloride (ArgMAm), or comprises four of those monomers. In some cases, the random polymer comprises at least one monomer of Formula I, chosen from dimethyl amino methacrylate (DMAEMA), 2-hydroxyethyl methacrylate, 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide.

In any of the above cases, the polymer sometimes has three monomers. In other cases, it has four monomers. In yet other cases, five monomers are used.

In some cases, the polymer comprises three monomers, wherein the three monomers are MMA, OEGMA, and either IBMA or BMA. In some cases, the three monomers are MMA, OEGMA, and IBMA. In some cases, the polymer comprises four monomers: MMA, OEGMA, IBMA or BMA, and DMAEMA or ArgMAm. In some cases the four monomers are MMA, OEGMA, IBMA or BMA, and DMAEMA. In some cases, the four monomers are MMA, OEGMA, IBMA or BMA, and ArgMAm. In some cases the four monomers are MMA, OEGMA, IBMA, and DMAEMA or ArgMAm. In some cases, the four monomers are MMA, OEGMA, IBMA, and DMAEMA. In some cases the four monomers are MMA, OEGMA, IBMA, and ArgMAm. In some cases, the polymer comprises a mix of both IBMA and BMA as well as MMA and OEGMA. In some cases, the polymer comprises a mix of both IBMA and BMA. In some cases, the polymer comprises more than one compound of Formula I, such as both DMAEMA and ArgMAm.

In other embodiments, the random polymer comprises two monomers. In some embodiments, the two monomers are BMA or IBMA and OEGMA. In some cases, the two monomers are IBMA and OEGMA. In some cases, the two monomers are BMA and OEGMA. In other cases, the two monomers are ArgMAm and OEGMA.

In any of the above cases, in some embodiments the polymer has an average molecular weight of from 3 to 20 kDa. In some embodiments, the polymer has an average molecular weight of from 3 to 15 kDa, from 5 to 20 kDa, from 5 to 15 kDa, from 5 to 10 kDa, from 5 to 8 kDa, from 10 to 20 kDa, from 7 to 10 kDa, from 8 to 10 kDa, from 10 to 12 kDa, from 12 to 15 kDa, or from 10 to 15 kDa.

When OEGMA is present, in some embodiments, the polymer comprises OEGMA with a molecular weight of from 300 to 1500 g/mol. In some such embodiments, the OEGMA may have a molecular weight of 300, 500, 750, 950, 1000, 1200, or 1500 g/mol. In some cases, it has a molecular weight of 500 g/mol (i.e., OEGMA 500).

In some embodiments, the polymer may comprise 20-50% MMA, 20-50% OEGMA, 5-25% IBMA by total weight of polymer. In some such cases, the polymer can be made from monomers consisting essentially of DMAEMA, IBMA, and OEGMA, for example, at 20-50% MMA, 20-50% OEGMA, 5-25% IBMA by total weight of polymer. In other cases, at least one additional monomer is also present, such as DMAEMA or ArgMAm. In some cases, the polymer may comprise 20-50% MMA, 20-50% OEGMA, 5-25% IBMA, and 5-25% DMAEMA. In some embodiments, polymers comprise DMAEMA or ArgMAm, IBMA, OEGMA, and MMA. In some cases, the monomers are present in a relative molar ratio of MMA, OEGMA, IBMA, and DMAEMA or ArgMAm as follows: (a) 5:2.5:2:0.5, (b) 2:5:2:1, (c) 5:2.5:1:1.5, (d) 4:4:1:1; (c) 3:4:1:2; (f) 2:4:1:3; (g) 2:3:1:4; or (h) 5:2:1:2. In some embodiments, the MMA, OEGMA, IBMA, and DMAEMA are in a molar ratio of 5:2.5:2:0.5, wherein the OEGMA has a molecular weight of 500 g/mol (i.e., is OEGMA 500), wherein the polymer comprises a molecular weight of from 3 to 15 kDa. In some embodiments, a polymer may be made from MMA, OEGMA, and IBBMA in a molar ratio of 5:4:1 or of 5:3:2 or of 3:5:2. And such a polymer may have a molecular weight of from 3 to 15 kDa. In some cases, the OEGMA may be OEGMA 500.

Certain particular exemplary polymers herein include, for example, Poly1, Poly3, Poly5, Poly7, Poly9, and Poly11, which comprise MMA, OEGMA, IBMA, and/or DMAEMA in the following molar ratios: Poly1:5:2.5:2:0.5, Poly 3:2:5:2:1, Poly 5:5:2.5:1:1.5, Poly11:4:4:1:1; Poly 9:3:5:2:0; and (f) Poly 7:5:3:2:0. In some such cases, the OEGMA has a molecular weight of 500 g/mol (i.e., is OEGMA 500), and the polymer comprises a molecular weight of from 3 to 15 kDa. In certain embodiments, a library comprises one or more of the above polymers Poly1, Poly3, Poly5, Poly7, Poly9, or Poly11 in a range of lengths, for example, corresponding to molecular weights of from 3 to 15 kDa. The corresponding polymers of different lengths/molecular weights in a library may be given designation letters A-H depending upon their average molecular weights from 3 to 15 kDa. Thus, for example, Poly1A comprises an average molecular weight of about 3 kDa and is made from MMA, OEGMA, IBMA, and DMAEMA in the following molar ratio: 5:2.5:2:0.5.

Further exemplary polymers herein include, for example, Poly20, Poly21, and Poly22, comprising MMA, OEGMA 500, IBMA, and/or ArgMAm, in molar ratios as follows: Poly20:5:2:1:2; Poly 21:0:5:4:1; and Poly 22:4:4:1:1.

The disclosure herein also encompasses particular polymer libraries that can be used as test excipients for particular protein formulations. For example, the disclosure encompasses an article of manufacture comprising a microwell plate or similar series of containers comprising aliquots of members of a polymer library, which can be added to a particular protein formulation in order to test their effects on the turbidity, viscosity, and other parameters of the formulation.

### III. Preparing Polymers and Polymer Libraries

The present disclosure also encompasses methods of preparing a polymer or polymer library as described herein. Acrylate and acrylamide polymers can also be prepared, for example, using reverse addition/fragmentation chain transfer (RAFT), free radical polymerization (FRP), and atom transfer radical polymerization (ATRP). See, e.g., S. Perrier, Macromolecules 50:7433-47 (2017); K. Matyjaszewski, Macromolecules 45 (10): 4015-39 (2012), for reviews of RAFT and ATRP methods, respectively. In some embodiments, polymers are prepared in a method that comprises exposing the chosen monomers to LED light in the presence of a zinc tetraphenyl porphyrin catalyst (ZnTPP). In some cases, a chain transfer agent may be added to the reaction mixture, for example, to control the polymerization process and the addition of the different monomers. The amount of chain transfer agent and/or catalyst may also aid in controlling the average length or average molecular weight, of the polymers. Examples of chain transfer agents include 2-cyano-2-propyl benzodithioate, 2-cyano-2-propyldodecyl trithiocarbonate, 4-((((2-carboxyethyl)thio) carbonothioyl)thio)-4-cyanopentanoic acid, 4-cyano-4-(phenylcarbonothioylthio) pentanoic acid, or 4-cyano-4-[(dodecylsulfanylthiocarbonyl) sulfanyl] pentanoic acid (CDTPA). (See, e.g., Perrier, cited above.) An exemplary method of making polymers and libraries herein is described, for instance, in Example 1 below. Methods of making random polymers and polymer libraries that are compatible with the polymers and libraries herein include those described, for example, in Gromley, A J et al., Angew. Chem. Int. Ed. 2018 vol. 57 (6), pp. 1557-1562, and in Ng. G. et al., Macromolecules 2018 vol. 51 (9) pp. 7600-7607.

### IV. Protein Formulations and Their Properties

Embodiments herein also include protein-containing formulations comprising the polymer excipients described herein. In some embodiments, the protein is an antibody, such as a monoclonal antibody, or antigen binding fragment thereof.

In some embodiments, the protein, such as an antibody, is present at a concentration of 20 mg/mL to 250 mg/mL, such as 20-200 mg/mL, 50-200 mg/mL, 50-150 mg/mL, 20-100 mg/mL, 50-100 mg/mL, 80-120 mg/mL, 100-200 mg/mL, or 150-200 mg/mL. In some embodiments, the protein formulation also comprises at least one buffer.

In some embodiments, the polymer excipient herein is present at a concentration of 0.001-1% w/v, such as 0.01-1% w/v, or 0.01-0.1% w/v. In some embodiments, the polymer excipient is present at 1-100 μM, such as 1-50 M, 10-100 μM, or 10-50 μM.

In some embodiments, the protein formulation also comprises a surfactant, such as polysorbate 20 (PS20), polysorbate 80 (PS80), pluronics, such as poloxamer 188 or pluronic F68, or Brij, as well as surfactants such as alkylglycosides, such as octyl maltoside, decyl maltoside, dodecyl maltoside, or octyl glucoside, a cholate surfactant such as CHAPS (3-[(3-cholamidopropyl) dimethylammonio]-1-propanesulfonate), SGH (sodium glycocholate hydrate), sodium taurocholate hydrate (STH), sodium cholate hydrate (SCH), SdTH, SdCH, ScdCH, or BigCHAP (N,N′-bis-(3-D-gluconamidopropyl) cholamide). In other embodiments, the protein formulation does not comprise a surfactant. If a surfactant is present, in some embodiments, it is present at a concentration of, for example, 0.001-1% w/v, such as 0.01-0.1% w/v.

In some embodiments, the protein formulation comprises a stabilizer such as an amino acid, sugar, sugar alcohol, or albumin. In some embodiments, no such stabilizer is present (i.e., the formulation does not comprise any amino acid, sugar, sugar alcohol, or albumin as a stabilizer).

In some formulations, the formulation comprises a salt, such as a sodium or potassium chloride, acetate, citrate, or phosphate salt, or such as arginine succinate, arginine hydrochloride or histidine hydrochloride. In other cases, the formulation does not comprise such a salt.

In some embodiments, the formulation does not comprise other polymers beyond one or more of the specific polymers described herein.

In some embodiments, the formulation consists essentially of the protein, a buffer, and the polymer herein. In some embodiments, the formulation consists essentially of the protein, a buffer, the polymer herein, and a surfactant. In some embodiments, the formulation consists essentially of the protein, a buffer, the polymer herein, a surfactant, and a salt. In some embodiments, the formulation consists essentially of the protein, a buffer, the polymer herein, and an amino acid, sugar, or sugar alcohol. In some embodiments, the formulation consists essentially of the protein, a buffer, the polymer herein, a surfactant, and an amino acid, sugar, or sugar alcohol.

In some embodiments, presence of the polymer significantly reduces the turbidity of the formulation, as measured at 600 nm (see Example 2 below) in comparison to a formulation that is otherwise identical but does not comprise the polymer. In some embodiments, presence of the polymer reduces the viscosity of the formulation at 25° C. and a 1000 l/s shear rate (see Example 3 below) in comparison to a formulation that is otherwise identical but does not comprise the polymer. In some embodiments, the turbidity and/or viscosity is reduced in comparison to a formulation in which the polymer is replaced by an equivalent concentration of a surfactant such as PS20.

### V. Testing of Protein Formulations

In some embodiments, different members of a polymer library, for example having different molecular weights or lengths and different ratios of monomers or different monomer choices may be placed into wells of microwell plates filled with a protein solution for testing, for example. Following such an approach, a series of polymers was obtained, for example, that have different ratios of monomers and/or different sets of monomers, as well as different lengths (corresponding to different molecular weights). In some cases, branched or straight chain polymers could also be compared. For example, using a 96-well or similar multi-well plate, multiple polymers can be tested for their effects on a particular protein formulation compared to control formulations.

In a microplate, for instance, the effects on protein stability, viscosity, turbidity, precipitation, microscale thermophoresis, and surface tension may be considered for polymers of different composition ratios or different monomers and for polymers of different lengths or molecular weights. Turbidity, for example, can be assessed by absorbance at 600 nm light wavelength. And exemplary assay is described in Example 2 below. Viscosity can be measured, for example, in a rheometer at a particular temperature and shear rate. (See Example 3 below.) The degree to which polymers interact with the protein can also be measured for various polymer or protein concentrations by microscale thermophoresis, as described in Example 4 below.

### EXAMPLES

The disclosure will be more fully understood by reference to the following examples. They should not, however, be construed as limiting the scope of the disclosure, however.

### Example 1. Materials and Methods

### A. Materials

Reagents were purchased from Sigma Aldrich, VWR, or Fisher Scientific and used without purification unless otherwise specified. Monomers were passed over basic alumina prior to polymerization to remove inhibitors. mAb-G was expressed in Chinese hamster ovary (CHO) cell lines. Sodium phosphate and histidine chloride buffers were prepared with compendia-grade (USP, NP, EP) chemicals and MilliQ water. mAb-G was concentrated and buffer exchanged using Millipore (Billerica, MA) Amicon Ultra centrifugation tubes (10 or 30 kDa molecular weight cutoff, MWCO). mAb-G solutions were filtered through Corning 0.22 μm polyethersulfone (PES) vacuum filters (Corning, NY) or MilliporeSigma MillexGV 0.22 μm polyvinylidene fluoride (PVDF) filters (Burlington, MA) prior to experiments. The Pierce Bradford assay kit was purchased from ThermoFisher Scientific. Jurkat cells were purchased from ATCC (TIB-152, clone E6-1, Lot #70044353).

### B. Polymer Library Synthesis

Libraries of polymers was synthesized using chain transfer (i.e., polymerization) reactions. Liquid monomers were passed over basic alumina to remove inhibitors of polymerization present in the stock solutions (e.g., hydroquinone monomethyl ether (also called p-methoxyphenol or MeHQ)). Stock solutions of monomer, chain transfer agent (CTA), and catalyst were prepared in DMSO. Respective volumes of stock solutions and DMSO were transferred to a 96-well plate, which was sealed with plate tape.

Polymers were composed of methyl methacrylate (MMA), oligo(ethylene glycol) methyl ether methacrylate (OEGMA), isobutyl methacrylate (IBMA), and dimethyl amino methacrylate (DMAEMA). Monomer compositions ranged from 5-50 mol % of polymer and monomer molecular weights ranged from 3-20 kDa.

Polymers were prepared at the following molar ratios of the monomers MMA:OEGMA:IBMA:DMAEMA, Poly1=5:2.5:2:0.5, Poly 3=2:5:2:1, Poly 5=5:2.5:1:1.5, Poly 7=5:3:2:0, Poly 9=3:5:2:0, Poly11=4:4:1:1) Other monomers used: (4-Hydroxyphenyl) methacrylamide (10-20 mol %), 2-Hydroxyethyl methacrylate (20-25 mol %), [2-(Methacryloyloxy)ethyl]dimethyl-(3-sulfopropyl) ammonium hydroxide (zwitterionic monomer, 10-25 mol %), plan to try arginine methacrylate, 2-Aminoethyl methacrylate hydrochloride.

In a first polymer library (in Example 2 below), polymers were synthesized to obtain a range of lengths (measured as average molecular weights), from 3 to 15 kDa. For example, Poly1, with a 5:2.5:2:0.5 molar ratio of MMA:OEGMA:IBMA:DMAEMA, was made in a range of average molecular weights from least to greatest designated A-H. Thus, for example, Poly1 was made with average molecular weights of 3 kDa (Poly1A), 7 kDa (Poly1D), 8.5 kDa (Poly1E), 10 kDa (Poly1F), with Poly1B and Poly1C having molecular weights between those of Poly1A and Poly1D and Poly1G having a molecular weight higher than that of Poly1F. A similar range of average molecular weight polymers was made for each of the polymers Poly3, Poly5, Poly7, Poly9, and Poly11.

To generate different polymer lengths, the molar ratio of the CTA to total monomers was controlled as follows, assuming 85% conversion of monomer to polymer: A 28:100; B 16:100, C 11:100, D 8:100, E 6:100, F 5:100, and G 4:100.

Individual reactions using different ratios of monomers and for production of polymers with different lengths or molecular weights were placed into individual wells of a 96-well plate.

C. Synthesis of N-(3-methacrylamidopropyl) guanidinium chloride (ArgMAm)

ArgMAm was synthesized via a slightly altered protocol previously reported (Funhoff et al., 2004). In a round bottom flask under N₂, N-(3-aminopropyl) methacrylamide hydrochloride (0.5 g, 2.8 mmol, 1 mol equiv.), 1H-pyrazole-1-formamidine monohydrochloride (0.41 g, 2.8 mmol, 1 mol equiv.), triethylamine (TEA) (0.9 mL, 6.7 mmol, 2.4 mol equiv.), and the polymerization inhibitor hydroquinone (5 mg) were dissolved in dimethylformamide (DMF) (8 mL). The reaction was stirred at room temperature (21° C.) for 24 h. The solution was poured into 50 mL cold diethyl ether and the oil was decanted. The oil was washed 2 times with 10 mL acetonitrile and once with 5 mL TEA, then centrifuged. The resulting solid was washed with 15 mL dichloromethane (DCM), centrifuged, decanted, and dried further by rotary evaporation yielding 147.14 mg (29% yield). 1H-NMR (400 mHz, DMSO-d6) δ ppm: 8.03 (t, J=5.6 Hz, 1H), 7.75 (t, J=5.9 Hz, 1H), 7.22 (s, 2H), 5.68 (p, J=1.1 Hz, 1H), 5.33 (p, J=1.6 Hz, 1H), 3.20-3.09 (m, 4H), 1.86 (dd, J=1.6, 0.9 Hz, 3H), 1.64 (p, J=6.8 Hz, 2H).

### D. Synthesis of Polymer Library by Photoinduced Electron Transfer Reversible Addition-fragmentation Chain Transfer (PET RAFT)

In a second polymer library (in Example 3 below), polymers of the same target length were synthesized using photoinduced electron transfer reversible addition-fragmentation chain transfer (PET RAFT). Each polymer in the library is intended to comprise the same length, as indicated by the identical volumes of chain transfer agent and polymerization catalyst in the polymerization reactions for all the polymers in the library.

Stock solutions of monomers (1 M), chain transfer agent (CTA) (0.2 M), and zinc tetraphenylporphyrin (ZnTPP) catalyst (8 mM) were prepared in dimethylsulfoxide (DMSO). Respective volumes of stock solutions and DMSO were transferred to a 96-well plate (Table 1), which was sealed with plate tape. Polymerization was initiated with a white 5k LED light and polymerization was allowed to proceed for 3-18 h. Polymerization was ended by removing the plate from the light source. Polymers were purified by microdialysis in a 96-deep well plate (Thermo Scientific, Pierce 3.5 kDa MWCO) against water for at least 2 days, changing dialysate 5 times. Representative ¹H-NMR Poly1 (400 mHz, DMSO-d6) δ ppm: 4.15-3.88, 3.82-3.59, 3.57-3.48, 3.48-3.37, 3.29-3.19, 2.26-2.11, 2.11-1.58, 1.52-1.37, 1.37-1.20, 1.20-1.07, 1.02-0.84, 0.84-0.60. Representative Mₙ Poly1 by GPC (polystyrene standards) 10.1 kDa, D=1.12.

The second polymer library of Example 3 was prepared as follows in Table 1:

TABLE 1
Volumes (μL) of Reagent Stock Solutions Used to Prepare the Second Library of Polymers.

| Polymer | CTA | ZnTPP | MMA | mOEGMA500 | IBMA | DMAEMA | ArgMAm | DMSO |
|---|---|---|---|---|---|---|---|---|
| Poly1 | 11 | 5 | 50 | 25 | 20 | 5 | 0 | 84 |
| Poly3 | 11 | 5 | 20 | 50 | 20 | 10 | 0 | 84 |
| Poly5 | 11 | 5 | 50 | 25 | 10 | 15 | 0 | 84 |
| Poly7 | 11 | 5 | 50 | 30 | 20 | 0 | 0 | 84 |
| Poly9 | 11 | 5 | 30 | 50 | 20 | 0 | 0 | 84 |
| Poly11 | 11 | 5 | 40 | 40 | 10 | 10 | 0 | 84 |
| Poly12 | 11 | 5 | 0 | 50 | 0 | 50 | 0 | 84 |
| Poly13 | 11 | 5 | 50 | 50 | 0 | 0 | 0 | 84 |
| Poly14 | 11 | 5 | 0 | 50 | 50 | 0 | 0 | 84 |
| Poly15 | 11 | 5 | 0 | 100 | 0 | 0 | 0 | 84 |
| Poly16 | 11 | 5 | 50 | 0 | 0 | 50 | 0 | 84 |
| Poly17 | 11 | 5 | 0 | 90 | 0 | 10 | 0 | 84 |
| Poly18 | 11 | 5 | 0 | 0 | 0 | 0 | 100 | 84 |
| Poly19 | 11 | 5 | 0 | 50 | 0 | 0 | 50 | 84 |
| Poly20 | 11 | 5 | 50 | 20 | 10 | 0 | 20 | 84 |
| Poly21 | 11 | 5 | 0 | 50 | 10 | 0 | 40 | 84 |
| Poly22 | 11 | 5 | 40 | 40 | 10 | 0 | 10 | 84 |

Polymerization was initiated with white 5k LED light. The polymerization reaction was allowed to proceed for 3 hours. Polymerization was ended by removing the 96-well reaction plate from the light source. Polymers were purified by microdialysis in a deep 96-well plate against water or buffer, for 2 days. The dialysate was exchanged 5 times.

Each polymer from the library, corresponding to a particular selection and ratio of monomers and a particular average molecular weight is thus present in a particular well of the 96-well plate, for subsequent testing.

### E. Nuclear Magnetic Resonance (NMR) Spectroscopy

Nuclear magnetic resonance (NMR) spectroscopy was performed on a 400 MHZ Bruker NMR instrument. Gel porosity chromatography (GPC) was performed on an Agilent 1200 series equipped with Wyatt Optilab T-rEX RI detector and a series of 3 Waters Styragel columns (HR 0.5, HR 2, HR 4) with tetrahydrofuran (THF) as the eluent at 1 mL/min and polystyrene conventional standards (Agilent EasiVial PS-M calibration standards). Protein concentration was determined by variable pathlength ultraviolet-visible (UV-vis) spectroscopy using a SoloVPE (C Technologies) connected to a Cary 60 UV-vis (Agilent) at 278 nm using the extinction coefficient. The absorbance precipitation assay and fluorescence measurements were performed on a Biotek Synergy Neo2 multi-mode plate reader.

### F. Microscale Thermophoresis (MST)

MST was performed on a Nanotemper Monolith NT.Automated instrument with Premium Coated capillaries at a polymer concentration of 15.5 μM and mAb-G concentrations of 2×10⁻⁷ to 1×10⁻⁴ M. Changes in polymer fluorescence upon heating in a capillary were measured.

### G. Isothermal Titration Calorimetry (ITC)

ITC was performed on a TA instruments Nano ITC with titration syringe volume of 50 μL and starting cell volume of 450 μL, with 25 injections of 2 μL. mAb-G (100 μM) and Poly1 (500 μM) were in 15 mM phosphate buffer pH 7.4. SCISSOR experiments were performed on a Sirius (now Pion) SCISSOR System.

### H. Dynamic Light Scattering (DLS)

DLS was performed on a Wyatt Dynapro plate reader in a half area 96-well plate at 25° C. with a polymer concentration of 0.1 w/v %. Live/Dead cell counting was measured using a Countess 3 FL (Invitrogen).

### I. Polymer Turbidity/Precipitation Assay

A sample monoclonal antibody protein for testing (e.g., mAb G) was buffer exchanged to 15 mM sodium phosphate buffer pH 7.4. In 96-well plates, water (control) or polymer/excipient stock solutions were added to achieve a final formulation comprising 1.6 μM polymer and 80 mg/mL protein and 15 mM sodium phosphate, pH 7.4.

Another sample of protein was diluted in the same volume phosphate buffer pH 7.4 but either with 100 mM sodium chloride, or without sodium chloride to act as a control sample. Control formulations comprising DMSO in place of sodium chloride and with buffer alone were also prepared.

The samples were pipetted into the 96-well plate to achieve a total volume=100 uL). The plates were incubated in ambient conditions at 4° C. for 18 hours, and turbidity was measured by absorbance at 600 nm. Measurements were recorded over a period of time starting after the above incubation or after 24 hours of incubation. Samples were removed from the plates after the assay and centrifuged and observed for any pellet formation in order to determine the extent to which presence of polymer allows protein to remain suspended in solution and prevents the protein from precipitating during the storage (as any pellets should be composed of protein without polymer).

The resulting turbidity measurements using mAb G are shown in Table 2 (Example 2) below, where solution turbidity was compared to mAb G in 100 mM NaCl (far right column showing p-value vs. mAb G w/salt). Samples contained 80 mg/ml mAb G in 15 mM phosphate buffer at pH 7.4 and contained 1.6 μM polymer where indicated. Measurements were made after samples had been incubated as described above for 18 hours at 4° C.

Turbidity measurements using mAb-G are described below in Example 3.

In some Examples described below, measurements were recorded at 25° C. or 37° C. instead of 4° C.

### J. Polymer Viscosity

Polymer viscosity was measured using a cone and plate rheometer with temperature set to 25° C. Polymer, excipient, or water (control) was added to each protein sample for final excipient concentrations that range from 0.02 and 0.1% (w/v).

### K. Surface Tension (ST)

A pendant drop tensiometer was used to measure surface tension of solutions with polymer or PS20 and a water control. Polymer Poly1F was prepared in water at 0.02% (w/v). PS20 was prepared in water at 0.05% (w/v). The tensiometer measures surface tension (ST) by recording images of each droplet that is formed from a test solution. The tensiometer software calculates ST based on the shape of each droplet. Each droplet was allowed to rest for 30 minutes after it is formed so that the measurements are recorded when the droplet is at a state of equilibrium. Measurements were carried out in triplicate and average ST was calculated.

### L. High Throughput Absorbance-based Precipitation Assays

mAb-G was buffer-exchanged into a 15 mM sodium phosphate buffer pH 7.4 and filtered. In a 96-well plate, water (control) or polymer stock solutions (20% w/v in water) were added to achieve 0.15% (w/v) solutions upon addition of protein. mAb-G was diluted for a final concentration of 80 mg/mL in the well with sodium phosphate buffer pH 7.4 containing NaCl for a final salt concentration of 150 mM in the well. Control without salt was also prepared in a similar manner. The solutions in tubes were thoroughly mixed on a roller and filtered. The solutions were pipetted into the 96-well plate containing polymer excipient stock solution aliquots or water for a total volume of 100 μL. The plate was sealed, incubated at 25° C., and turbidity was measured by absorbance at 600 nm every 2 hours for 46 hours.

### M. Low-throughput Eppendorf LoBind Tube Precipitation Assays

mAb-G was buffer-exchanged into a 15 mM sodium phosphate buffer pH 7.4 and filtered. mAb-G was diluted for a final concentration of 80 mg/mL in the tube with sodium phosphate buffer pH 7.4 containing NaCl for a final salt concentration of 150 mM in the well. Control without salt was also prepared in a similar manner. The solutions in tubes were thoroughly mixed on a roller. In a series of tared Eppendorf LoBind tubes, polymer stock or water (control) and mAb-G solution were added for a total volume of 200 μL. The tubes were incubated at 37° C. in an incubator and periodically removed for analysis. Tubes were centrifuged at 3200×g for 2 minutes, decanted, and lyophilized. Soluble protein concentration was measured by SoloVPE as described in the analytical techniques section and the weight of the precipitate after lyophilization was measured.

### N. FITC Labeling of Poly1

FITC-maleimide was conjugated to the polymer by aminolysis of the trithiocarbonate followed by maleimide-thiol coupling. Poly1 (200 mg/mL, 50 μL) was dissolved in water in an Eppendorf tube. Tris(2-carboxyethyl) phosphine (TCEP) (30.71 mg) dissolved in 100 μL water was added, followed by 20 μL of ethanolamine. The reaction was mixed on a shaker for 2 h. FITC-maleimide (3.05 mg, 5 equiv) dissolved in 100 μL 15 mM phosphate pH 7.4 buffer and 80 μL DMF, and was added to the reaction mixture. The reaction was mixed an additional 2 h, then the polymer was purified by dialysis (Pierce Slide-A-Lyzer dialysis cassettes, 3 kDa MWCO) against water for 2 days. Degree of labeling (DOL) was calculated using the molar extinction coefficients of FITC (494 nm, 68,000 M⁻¹cm⁻¹) and Poly1 (420 nm, 69,400 M⁻¹cm⁻¹), correction factor 0.3, and the absorbance of the labeled polymer at wavelengths 494 and 420 nm (Equation 1).

$$DOL = \frac{{A}_{494}}{{ε}_{FITC} * \frac{( {A}_{420} - ( {A}_{494} * 0.3 )}{{ε}_{Poly 1}}} . \qquad Equation 1$$

Where A₄₉₄ and A₄₂₀ are the absorbance at 494 and 420 nm, ε_{FITC} ε_{Poly1} are the molar extinction coefficients for FITC and Poly1 at each respective wavelength. DOL 0.23568.

### O. SCISSOR System Solubility and Pharmacokinetic (PK) Assays

SCISSOR experiments were performed with minor alterations as described in the literature (Bown et al., 2018a). The chamber was filled with 300 mL SCISSOR buffer containing 6.4 g NaCl, 0.09 g MgCl₂·6H₂O, 0.4 g KCl, 0.2 g CaCl₂) and 2.1 g NaHCO₃ per 1 L Milli-Q water, which was equilibrated to 34° C. and maintained at pH 7.4 with CO₂. The SCISSOR cartridge was filled with 5 mL of a solution of 6.25 mg/mL 1.38 MDa hyaluronic acid (HA) dissolved in phosphate buffered saline (PBS) pH 7.4 and kept ˜1 cm above the bottom of the cartridge holder (above the clip) so that transmittance at the very bottom could be detected. A 1 ml syringe with a 25G needle was used to inject 0.2 mL mAb-G (80 mg/mL) into the chamber at a constant rate over approximately 20 s. The autosampler was set to collect samples at 5, 10, 15, 20, 25, 30, 40, 50, 60, 90, 120, 150, 180, 210, 240, 270, 300, 330, 360, and 390 min after injection. Offline analysis of the collected samples was completed by the Bradford assay for mAb-G or by fluorescence measurements (100 μL samples in black 96-well plate, λₑₓ=490 nm, λₑₘ=555 nm) for the FITC-Poly1 via standard curves for each method. The Bradford assay was performed as described by the manufacturer for the micro microplate procedure (96-well plate, 150 μL sample, 150 μL reagent, A₅₉₅ₘₘ). Pictures of the cartridge through the window of the chamber to visualize any precipitates were also taken during the experiment.

### P. Intrinsic Fluorescence

In a 96-well plate, 100 μL of mAb-G stock at 1 mg/mL and 100 μL polymer stock at various concentrations (5-100 mol equiv) both in 15 mM phosphate buffer pH 7.4 were mixed (n=3 for each polymer concentration). Controls of polymer without buffer and of buffer alone were also included and used to subtract as background. The plate was briefly centrifuged (20 s, approx. 150×g) and 50 μL each solution was transferred to a black 384-well plate. The plate was briefly centrifuged (20 s, approx. 150×g) and fluorescence was measured (λₑₓ=295 nm, λₑₘ=340 nm). Additionally, absorbance was measured at 295 nm and 340 nm. Corrections and control experiments were included as described in the literature (van de Weert & Stella, 2011a). In summary, the absorbance measurements were used to correct for the inner filter effect (Equation 2).

$${F}_{obs} = {F}_{corr} * {10}^{- \frac{{A}_{ex} * {d}_{ex}}{2} - \frac{{A}_{em} * {d}_{em}}{2}} . \qquad Equation 2$$

Where F_{obs} and F_{corr} are the measured and corrected fluorescence respectively, λₑₓ and λₑₘ are the absorbance values at the excitation and emission wavelengths, and dₑₓ and dₑₘ are the pathlength (in cm). The pathlength in the well (0.2 cm) was estimated using the well dimensions and volume of sample in the wells.

Fluorescence decrease was also confirmed not to be due to collisional quenching by calculating the quenching constant, k_{q}, using the Stern-Volmer equation (Equation 3).

$$\frac{{F}_{0} - F}{F} = {k}_{q} * τ * [ L ] . \qquad Equation 3$$

Where F is the fluorescence at a given polymer concentration [L], Fₒ is the fluorescence without polymer, and t is the fluorescence lifetime of tryptophan (1-10 ns). The quenching constant was 1.02×10¹² M⁻¹s⁻¹, larger than 2× 10¹⁰ M⁻¹s⁻¹ for a collisional quenching process.

### Q. Cell Viability in Jurkat Cells by Trypan Blue Assay

Jurkat cells were passaged 4 times in RPMI 1640 media (10% FBS, 1% PenStrep), incubating at 37° C., 95% humidity, and 5% CO₂. The cells were centrifuged and suspended at 4.4×10⁶ cells/mL in media. In a sterile 24-well plate, 100-L cell suspension was added to each well for a final cell concentration of 8×10⁵ cells/mL. Poly1 was diluted in media and sterile-filtered. To each well, 400 L of Poly1 solutions or media was added (n=4). The cells were incubated for 24 h and cell viability was assessed by the trypan blue assay. Trypan blue was added 1:1 to suspended cells and cells were counted for % Live/Dead. Cell viability was calculated by normalizing to the control without additive

### Example 2. Library Comprising Polymers of Different Lengths

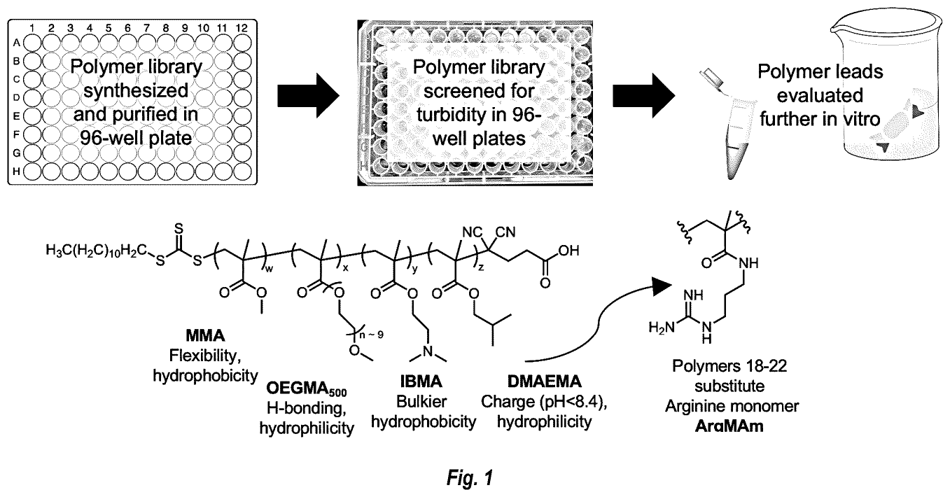

Several monomers targeting various intermolecular interactions were selected (see FIG. 1). Two hydrophobic monomers, methyl methacrylate (MMA) and isobutyl methacrylate (IBMA) were selected to imbue hydrophobic interactions with slightly different molecular weights. Two hydrophilic monomers were chosen. Oligo (ethylene glycol) methyl ether methacrylate 500 Da (OEGMA₅₀₀) was selected as a hydrogen bond acceptor and to convey aqueous solubility. Dimethylaminoethyl methacrylate (DMAEMA) was chosen as a positively charged monomer to target interactions with negative patches in the mAbs. From these monomers, polymers Poly1, Poly3, Poly5, Poly7, and Poly9 were prepared, each in different lengths, designated by a letter after the polymer name (e.g., Poly1A-G, Poly3A-G, etc.) with letters running from shortest to longest.

### A. Polymer Turbidity

Polymer turbidity was measured as described in Example 1 above. As shown in Table 2, the absorbance at 600 nm (A₆₀₀) was significantly reduced compared to the protein in 100 mM NaCl by either removing the salt or replacing it with DMSO, or by addition of particular polymer species. Entries with an asterisk (*) are relevant controls and samples that were statistically significantly different from positive control, specifically Poly1D, Poly1F, and Poly7D.

TABLE 2
Polymer Turbidity.

| Sample | A₆₀₀ | Error | P (vs mAb G w/ salt) |
|---|---|---|---|
| mAb G 100 mM NaCl* | 0.236 | 0.012 | |
| mAb G + DMSO* | 0.007 | 0.008 | 0.0083 |
| Buffer* | 0.106 | 0.007 | <0.0001 |
| mAb G no salt* | 0.112 | 0.005 | <0.0001 |
| Poly1A | 0.249 | 0.138 | |
| Poly1B | 0.222 | 0.008 | |
| Poly1C | 0.218 | 0.019 | |
| Poly1D* | 0.201 | 0.010 | 0.0002 |
| Poly1E | 0.213 | 0.021 | 0.1991 |
| Poly1F* | 0.207 | 0.018 | 0.0405 |
| Poly1G | 0.307 | 0.109 | |
| Poly7A | 0.241 | 0.011 | |
| Poly7B | 0.233 | 0.009 | |
| Poly7C | 0.241 | 0.005 | |
| Poly7D* | 0.192 | 0.006 | 0.0047 |
| Poly7E | 0.248 | 0.002 | |
| Poly7F | 0.276 | 0.012 | |
| Poly7G | 0.234 | 0.011 | |
| Poly9A | 0.241 | 0.053 | |
| Poly9B | 0.359 | 0.145 | |
| Poly9C | 0.495 | 0.332 | |
| Poly9D | 0.609 | 0.309 | |
| Poly9E | 0.223 | 0.170 | |
| Poly9F | 0.429 | 0.160 | |
| Poly9G | 0.300 | 0.043 | |

A = avg. 3 kDa; B and C = 3-7 kDa; D = avg. 7 kDa; E = avg. 8.5 kDa; F = avg. 10 kDa; G = >10 kDa.

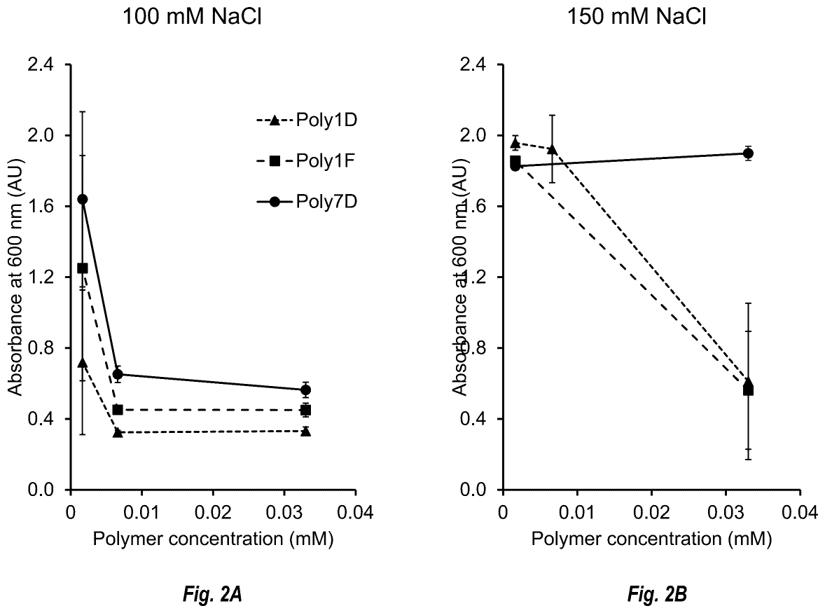

Further data are shown in FIGS. 2A-4 and 7A-B. FIGS. 2A-2B show absorbance at 600 nm in a formulation comprising 80 mg/mL mAb G and 15 mM phosphate buffer at pH 7.4, and either 100 mM NaCl (FIG. 2A) or 150 mM NaCl (FIG. 2B), in the presence of increasing concentrations of Poly1 species of two different molecular weights, Poly1D (avg. 7 kDa), Poly1F (avg. 10 kDa), as well as a species of Poly7, Poly7D (avg. 7 kDa), over a concentration range of 0-0.4 mM polymer (specifically with polymer concentrations of 0, 1.6, 6.6, or 33 μM; about 0.0014, 0.006, or 0.03% w/v). As shown in the figure panels, all polymers reduced the absorbance at 600 nM in the 100 mM NaCl formulations, but only the two Poly1 species reduced the absorbance at 600 nm in the 150 mM NaCl formulation.

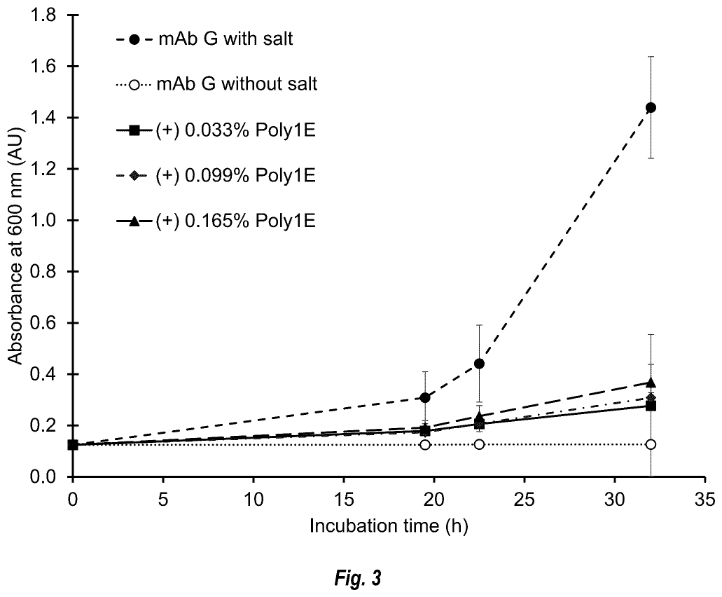

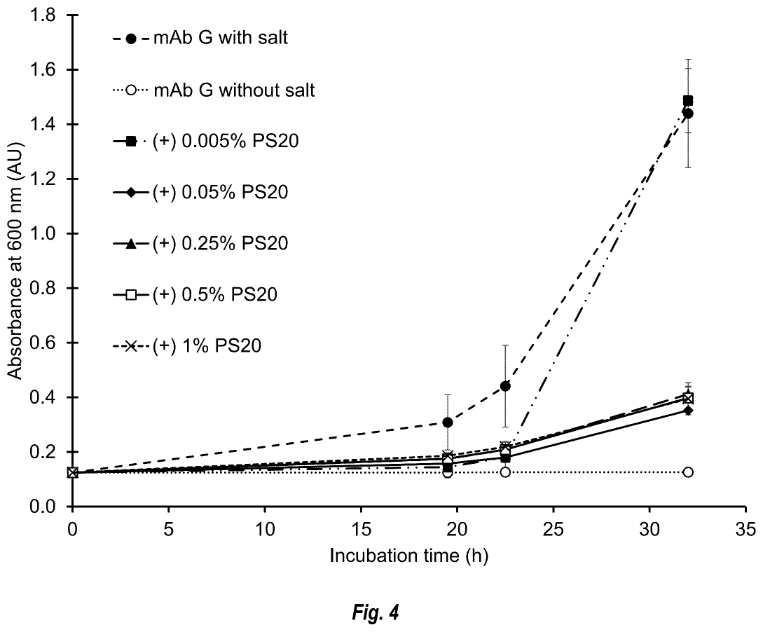

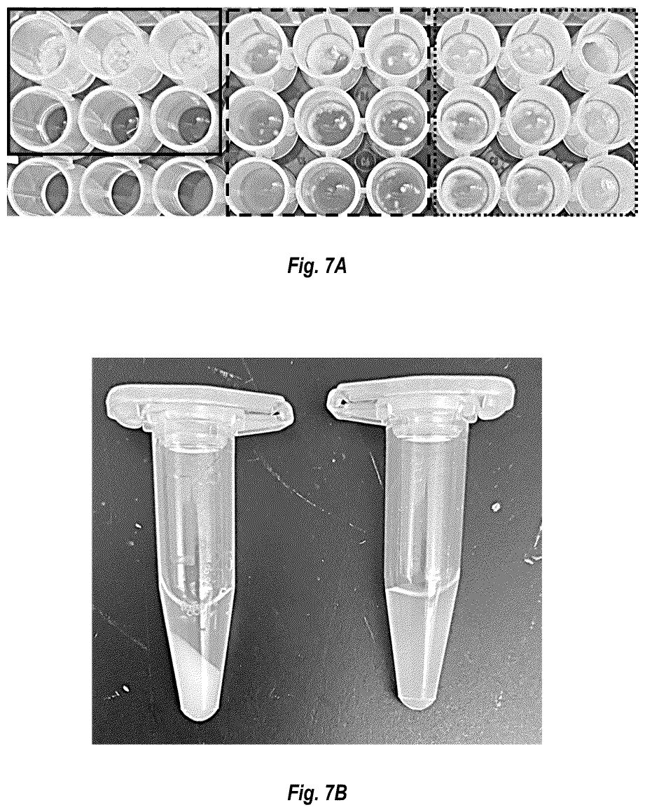

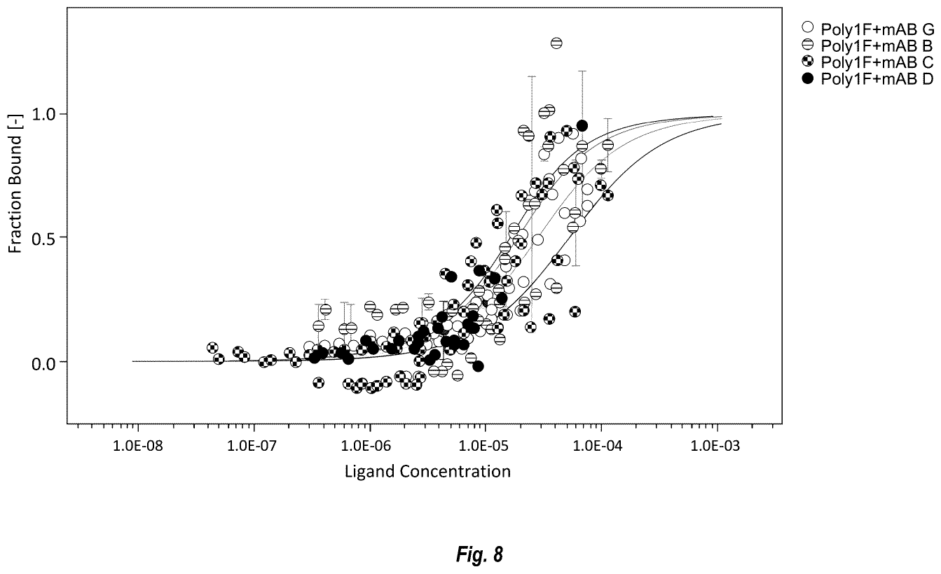

FIG. 3 shows the precipitation kinetics of the formulations above with 150 mM NaCl and three different concentrations of Poly1E (avg. 12 kDa). All three concentrations of Poly1E were able to prevent significant increase in absorbance at 600 nm over an incubation time of up to 32 hours at 23° C. FIG. 4 shows the precipitation kinetics of formulations above in the presence of polysorbate 20 (PS20) at several different concentrations, in lieu of the polymer excipients. As shown in FIGS. 3-4, presence of Poly1E led to a lower increase in absorbance over time, as compared increase in absorbance of a solution with PS20 or a control solution without additive. FIG. 7A shows images of aliquots of the mAb G formulation, comprising 80 mg/mL mAb G, 15 mM phosphate buffer at pH 7.4, 150 mM NaCl, and with no additional excipients (left), increasing concentrations of Poly1E (middle), and increasing concentrations of PS20 (right). FIG. 7B shows an aliquot of the formulation comprising PS20 excipient (left) or Poly1E excipient (right) after centrifugation to separate precipitant. As is evident from FIG. 7B, there is significantly more insoluble precipitate after centrifugation in the formulation comprising PS20 than in the formulation comprising Poly1E.

### B. Polymer Viscosity

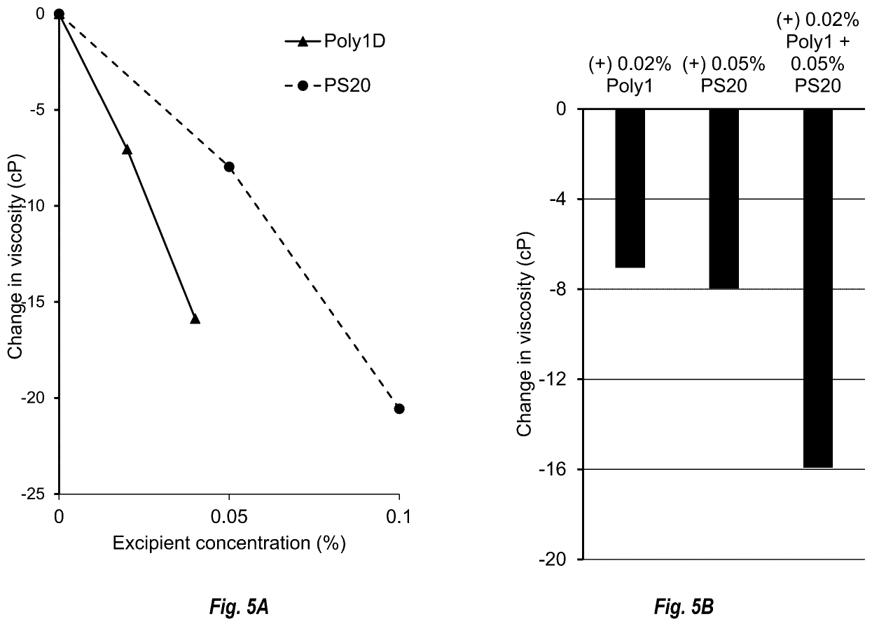

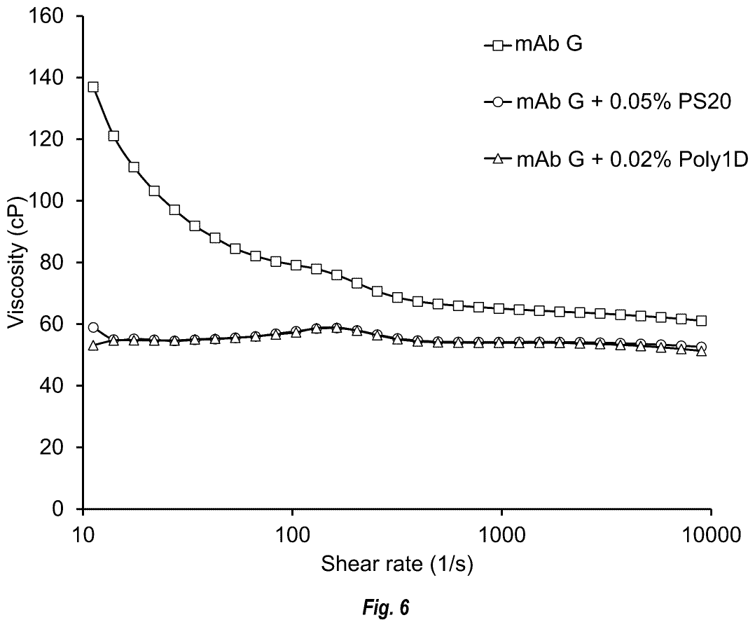

Polymer viscosity was measured as described in Example 1 above. Viscosity data are provided in FIGS. 5A-6. FIG. 5A shows change in viscosity (in cP) for a mAb G solution at 195 mg/mL mAb G in 15 mM histidine chloride buffer at pH 5.5 at 25° C. with either Poly1D or PS20 excipients at increasing concentrations of 0-0.1% w/v of the formulation. Single point viscosity was measured at 1000 l/s shear rate. As shown in the FIG. 5A, Poly1D was more effective than PS20 at reducing viscosity. FIG. 5B shows the degree of reduction in viscosity upon addition of 0.02% w/v Poly1D alone, 0.05% w/v PS20 alone, or a combination of both 0.02% Poly1D and 0.05% PS20. While the change is similar for each polymer alone, addition of the two significantly further reduced viscosity.

FIG. 6 shows viscosity dependence of mAb G at 194 mg/mL in 15 mM histidine chloride buffer at pH 5.5 at 25° C. on shear rate, for a shear rate ramp of from 10 to 10000 l/s, to evaluate the effect of shear rate on viscosity. As shown in the figure, without either PS20 or Poly1D, the mAb G formulation shows decreasing viscosity upon increasing shear rate between about 10 and 100 l/s. The presence of either PS20 or Poly1D eliminates this dependence, indicating that both of these excipients are surface active. A shear rate of 1000 l/s was chosen for the next set of experiments.

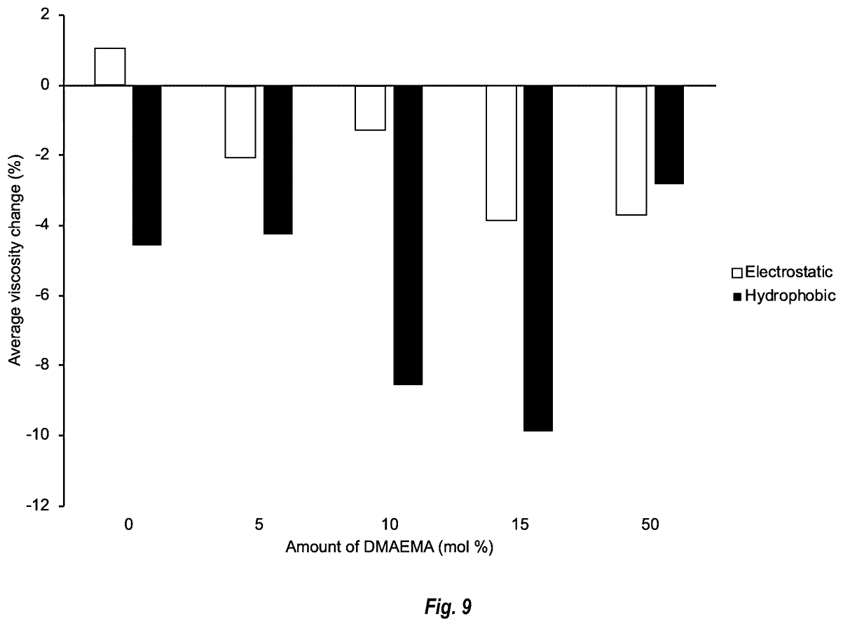

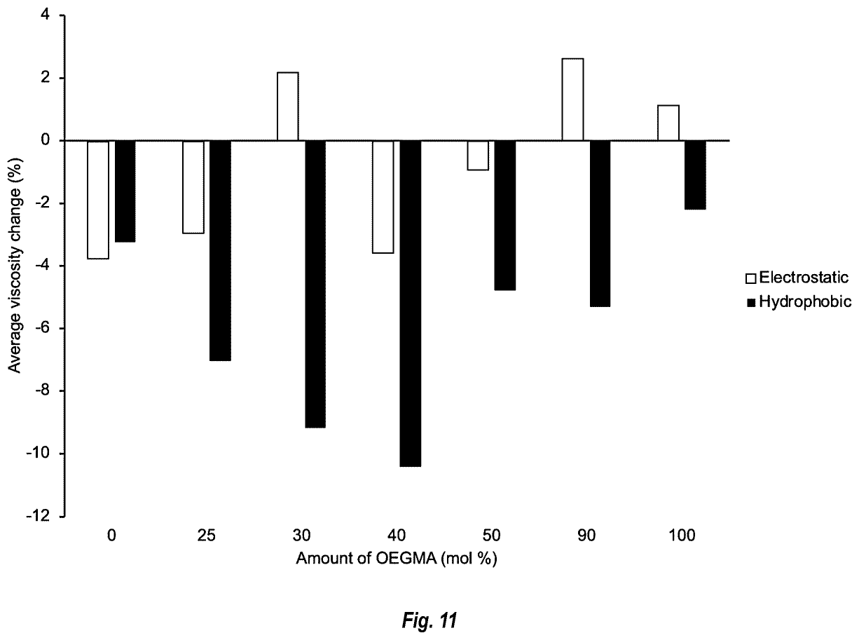

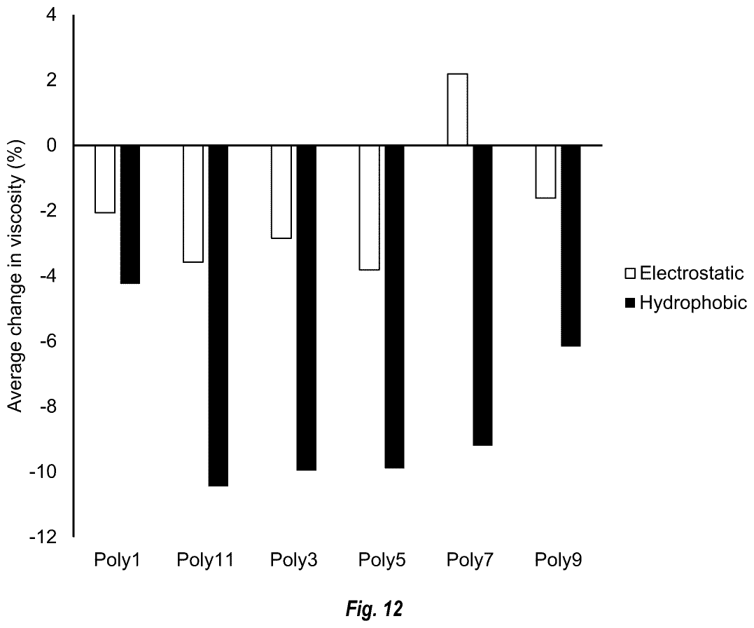

The effect of excipients on viscosity of a variety of protein formulation samples is shown in Tables 3 and 4 and FIGS. 9-12. The concentration of each of sample monoclonal antibody proteins mAbs G, B, C, and E and a Fab fragment Fab G ranged from 194 to 207 mg/mL in 15 mM histidine chloride pH 5.5. The concentration of each polymer from the library was 33 μM (i.e., 0.03%).

The antibody species tested are as follows: mAb G, mAb B, mAb C and mAb E are monoclonal IgG antibodies, mAb D is a bispecific scFv monoclonal antibody, and Fab G is a Fab fragment.

Viscosity was measured with and without 0.15% (w/v) polymers in the library in 30 mM HisCl. In FIGS. 9-12, average relative % change in viscosity (% change normalized by initial viscosity w/o additive) with polymer was plotted by polymer composition (for several monomers), and polymer identity.

mAbs are classified as electrostatic or hydrophobic based on mechanism driving high viscosity. For solutions of mAb G, mAb E, and mAb C, for example, hydrophobic protein-protein interactions are thought to drive changes in viscosity, as indicated in Table 3. For solutions of mAb B and Fab G, for example, electrostatic interactions are thought to drive changes in viscosity, as indicated in Table 4. In general, the tables show a greater decrease in viscosity in the presence of polymer excipient where the viscosity is driven by hydrophobic interactions.

TABLE 3
Polymer Viscosity Driven by Hydrophobic Interactions.

| Sample | Viscosity (cP) | Viscosity change (cP) | Fold Change |
|---|---|---|---|
| mAb G | 133.80 | | |
| mAb G + 1F | 117.06 | −16.74 | 1.14 |
| mAb G + 3F | 120.21 | −13.59 | 1.11 |
| mAb G + 5F | 123.60 | −10.20 | 1.08 |
| mAb G + 11F | 122.48 | −11.32 | 1.09 |
| mAb E | 55.73 | | |
| mAb E + 1F | 33.26 | −22.47 | 1.68 |
| mAb E + 3F | 38.70 | −17.03 | 1.44 |
| mAb E + 5F | 37.27 | −18.46 | 1.50 |
| mAb E + 11F | 36.32 | −19.41 | 1.53 |
| mAb C | 181.48 | | |
| mAb C + 1F | 53.53 | −127.95 | 3.39 |
| mAb C + 3F | 133.61 | −47.87 | 1.36 |
| mAb C + 5F | 37.08 | −144.40 | 4.89 |
| mAb C + 11F | 30.71 | −150.77 | 5.91 |

TABLE 4
Polymer Viscosity Driven By Electrostatic Interactions.

| Sample | Viscosity (cP) | Viscosity change (cP) | Fold Change |
|---|---|---|---|
| mAb B | 70.74 | | |
| mAb B + 1F | 55.75 | −14.99 | 1.27 |
| mAb B + 3F | 50.29 | −20.45 | 1.41 |
| mAb B + 7F | 43.93 | −26.81 | 1.61 |
| mAb B + 9F | 42.08 | −28.66 | 1.68 |
| mAb B + 11F | 38.45 | −32.29 | 1.84 |
| Fab G | 51.04 | | |
| Fab G + 1F | 35.37 | −15.67 | 1.44 |
| Fab G + 7F | 45.20 | −5.84 | 1.13 |
| Fab G + 11F | 36.17 | −14.87 | 1.41 |

### C. Microscale Thermophoresis (MST)

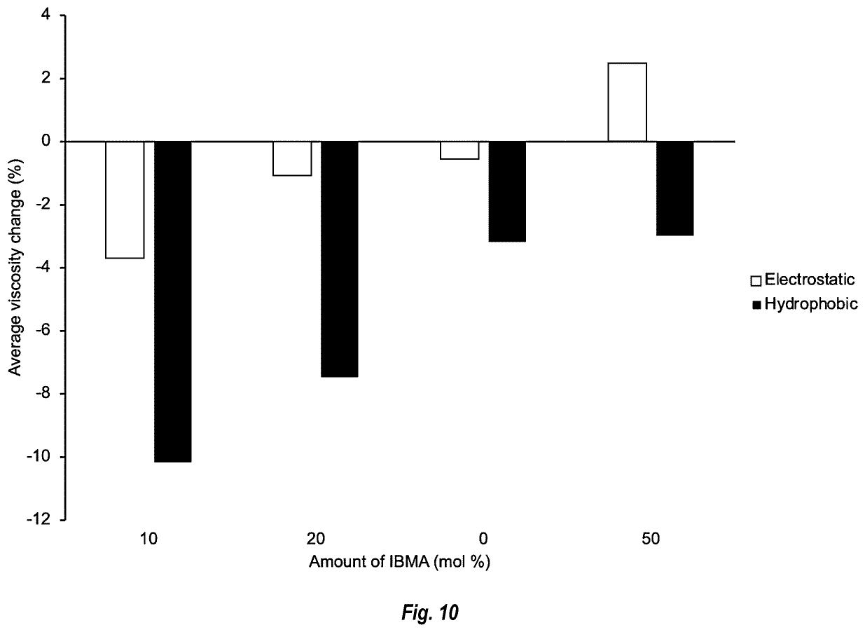

Molecular interaction between polymer and several proteins was measured using microscale thermophoresis (MST), a method used to calculate the fraction of protein bound to polymer. MST was performed as described in Example 1 above. As shown in FIG. 10, four different proteins in formulations comprising Poly1F showed different degrees of molecular interactions with increasing polymer concentration.

### Example 3. Library Comprising Series of Polymers of the Same Length and Diverse Monomer Contents

A second polymer library was prepared as described above in Example 1 in a 96-well plate using photoinduced electron transfer reversible addition-fragmentation chain transfer (PET RAFT) (Table 1; FIG. 1). This technique has been used previously to prepare polymers in a high throughput manner without rigorous degassing required for other controlled radical polymerization techniques and can be achieved at room temperature with visible light initiating polymerization through a photocatalyst (Gormley et al., 2018; Ng et al., 2018).

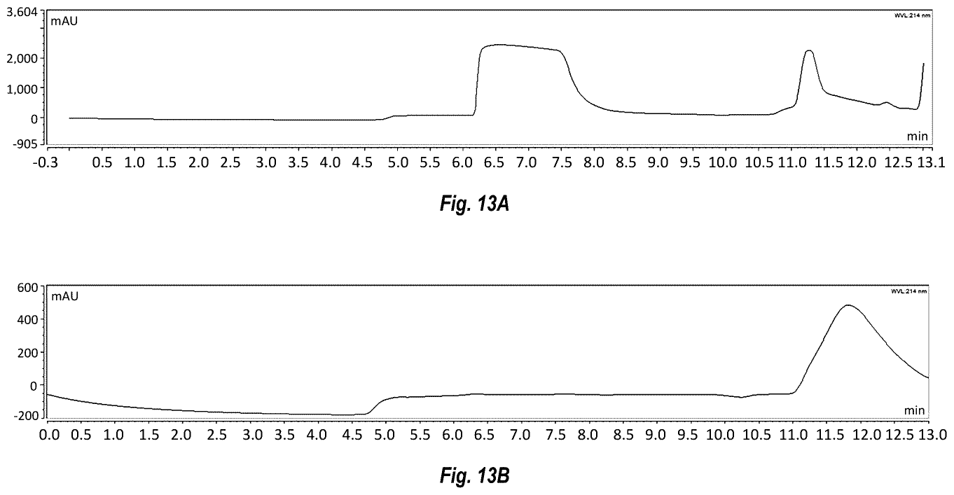

Again, three hydrophilic monomers were chosen, one neutral and two charged. Oligo (ethylene glycol) methyl ether methacrylate 500 Da (OEGMA500) was selected as a hydrogen bond acceptor and to convey aqueous solubility. Dimethylaminoethyl methacrylate (DMAEMA) and arginine mimicking monomer N-(3-methacrylamidopropyl) guanidinium chloride (ArgMAm) were chosen as positively charged monomers at pH 7-4 (Funhoff et al., 2004; Laaser et al., 2015; Lee et al., 2011) to promote electrostatic interactions with negative patches on the mAb surface and/or increase electrostatic repulsion between mAbs. Additionally, ArgMAm was selected as an arginine mimic because arginine is an excipient commonly used as a buffer component and arginine reduces viscosity when added as an excipient in many protein formulations (Shukla & Trout, 2010; Sudrik et al., 2017). This library was designed to explore compositions of MMA, OEGMA₅₀₀, IBMA, and DMAEMA or ArgMAm while targeting solubility of the final polymers in aqueous solution with 2-4 monomers included in the composition. Polymerization was confirmed by GPC with 3 randomly selected polymers. Molecular weights of the polymers described herein were Mₙ of ˜10 kDa (representative Poly1 Mₙ 10.1 kDa, [symbol] =1.12, Poly7 Mₙ 11.3 kDa, [symbol] =1.22, Poly22 Mₙ 12.3 kDa, [symbol] =1.11). Polymers were purified by microdialysis in the same 96-well format into water for 3 days and elimination of DMSO and reactant impurities was confirmed by HPLC (FIG. 13A-B).

### A. Polymer Turbidity

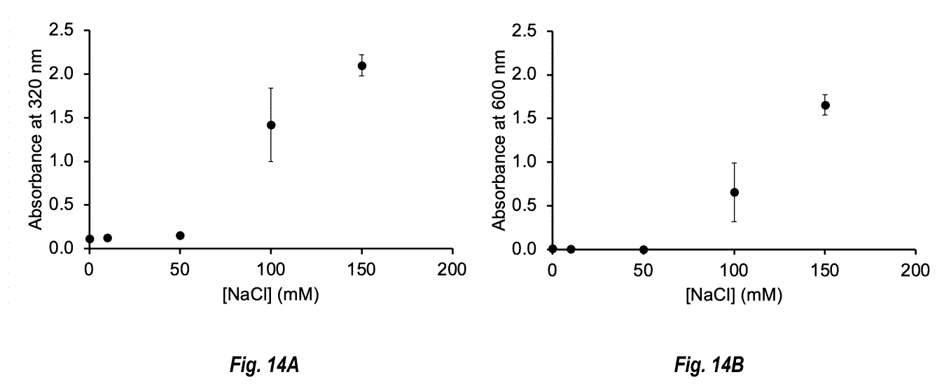

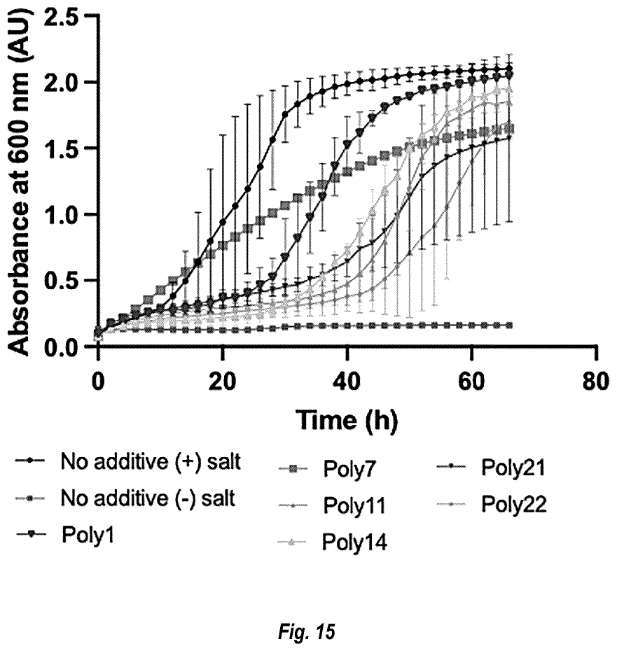

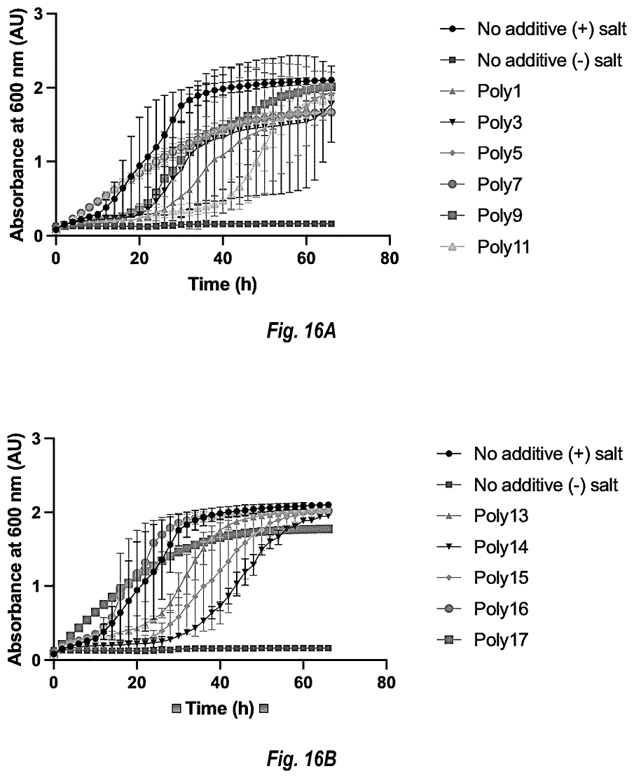

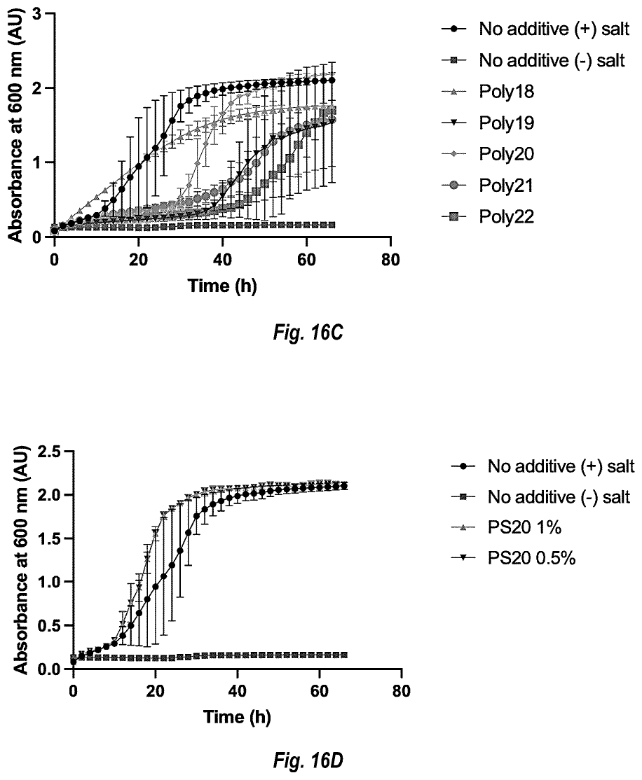

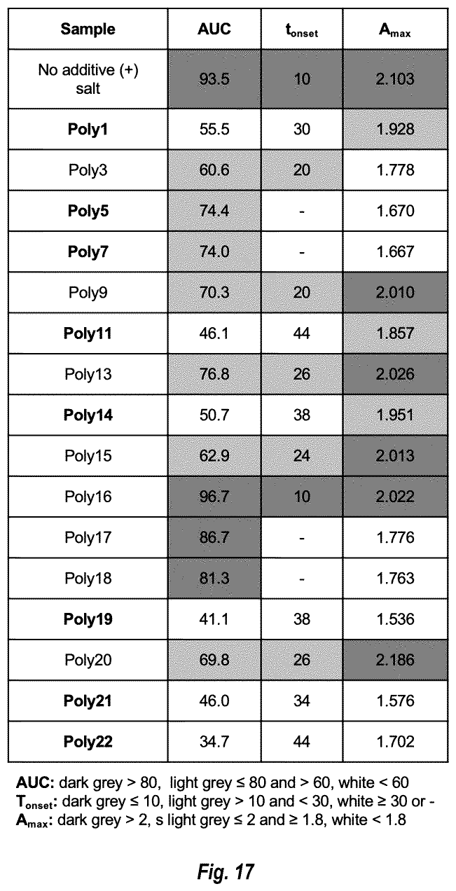

A high throughput turbidity screening assay based on visible light absorbance (described above in Example 1) was used to investigate the effect of polymers from the second library on the precipitation of mAb-G. In preliminary experiments, the best sensitivity to precipitation with minimal background from the protein was observed by measuring absorbance at 600 nm (FIG. 14A-B). The protein was buffer exchanged to 15 mM phosphate buffer pH 7.4 because the protein does not precipitate at low ionic strength at this pH. 150 mM of sodium chloride (NaCl) and 0.15% heteropolymer excipient stock solutions for were added. Absorbance was monitored at 600 nm to quantify the precipitation of the mAb. Plots of absorbance over incubation time are shown in FIGS. 15 and 16A-D. Three attributes of the precipitation kinetics were quantified: precipitation onset time (tₒₙₛₑₜ), total area under the curve (AUC), and absorbance maximum (Aₘₐₓ). Qualitative ranking compared to the control highlighted promising polymer candidates (FIG. 17). Several polymers with two or more white or no light grey attributes were selected as leads for further analysis.

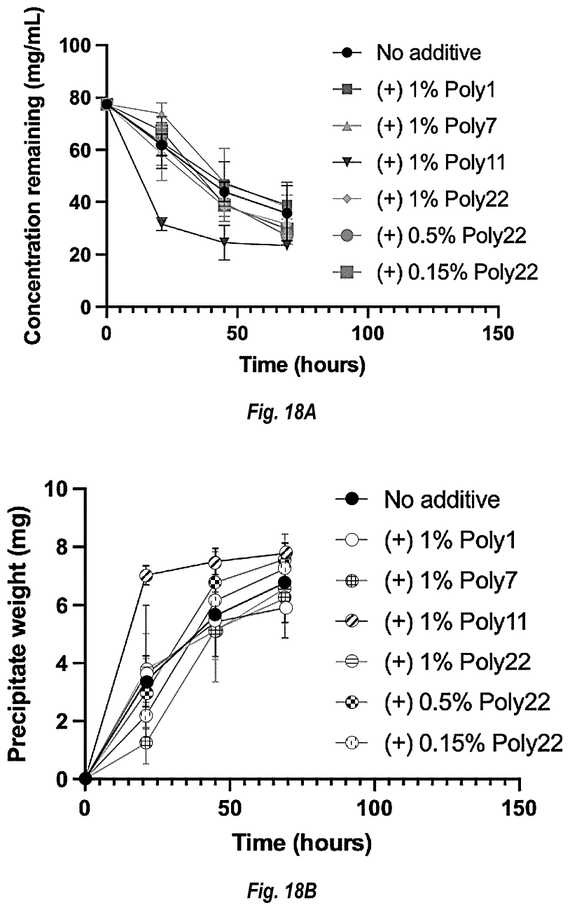

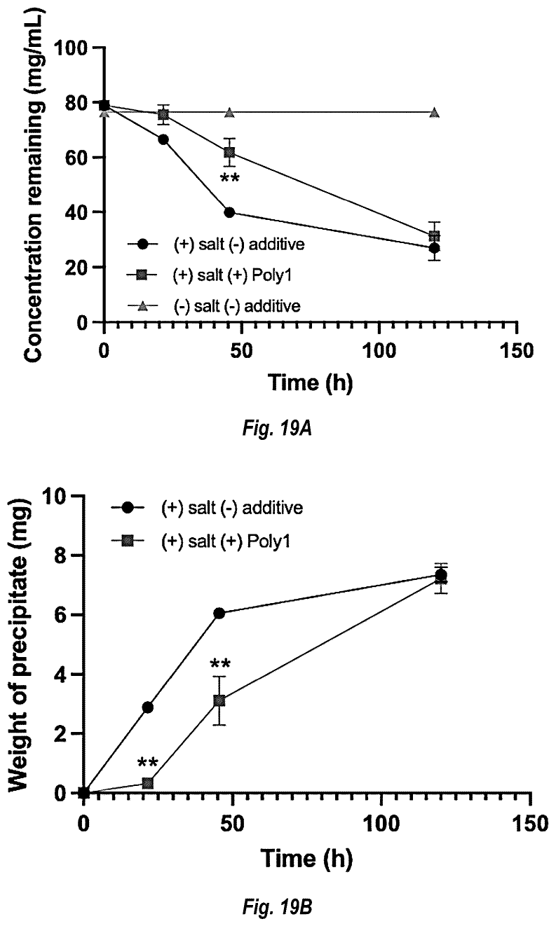

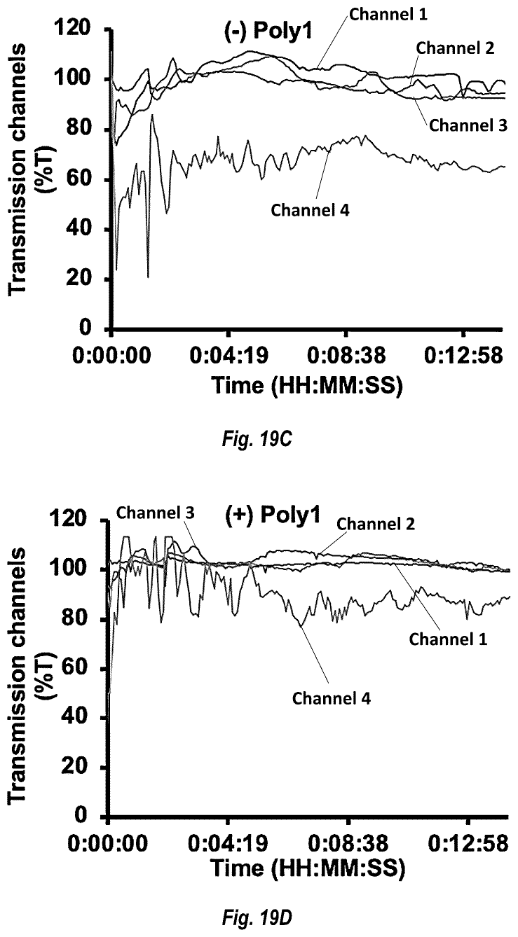

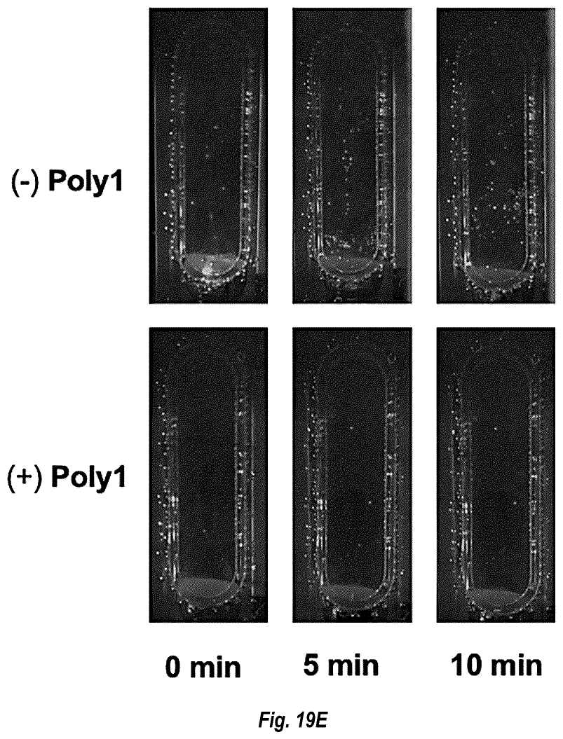

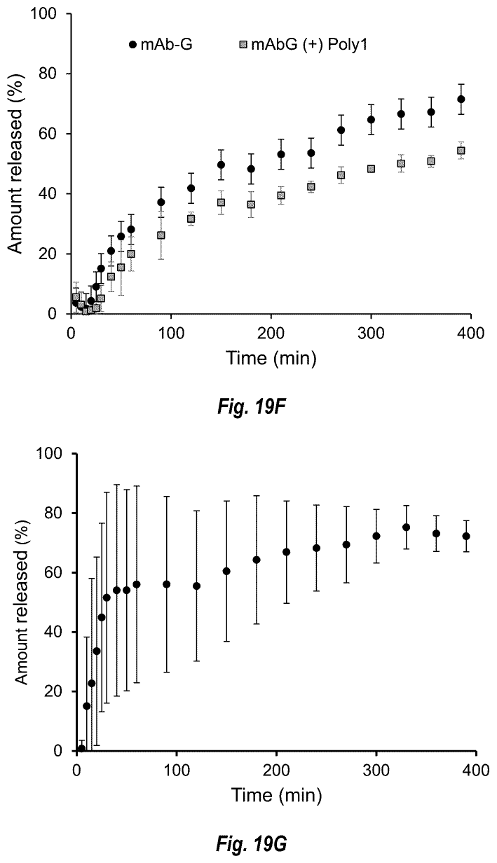

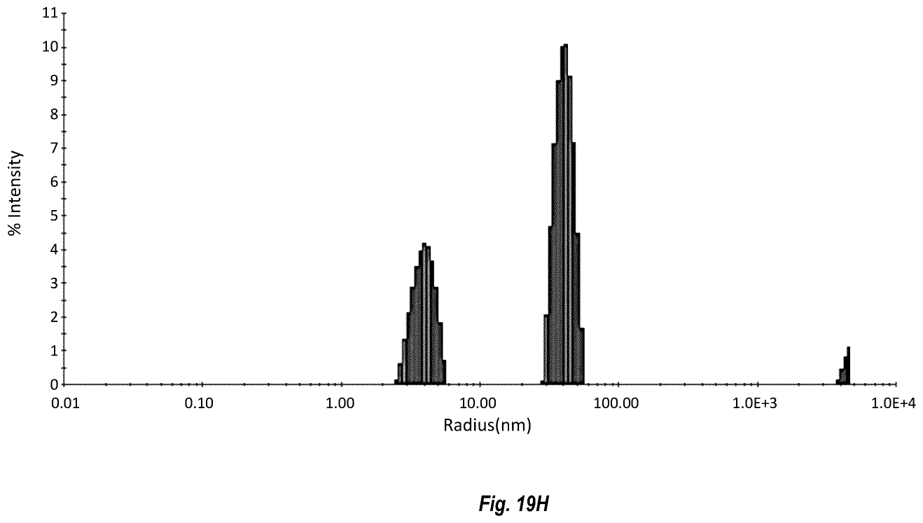

Characterization of precipitation under relevant temperature conditions was completed through a lower-throughput, higher volume method in Eppendorf tubes to mitigate artifacts from evaporation observed in plate-based UV-vis assay at temperatures above 25° C. (data not shown). To a series of tubes, mAb-G formulated in 15 mM phosphate buffer pH 7.4, NaCl solution, and polymer solutions were added and the concentration of mAb-G in the supernatant after centrifugation and the weight of the dried pellet were measured at prescribed time intervals. Surprisingly, most of the polymers selected from the screen did not improve the solubility of mAb-G (FIGS. 18A-B). In one case, at higher concentration of polymer (Poly11), the precipitation was faster than without additives. The change in temperature and polymer concentration may have played a significant role in how the excipients affect the precipitation kinetics of the mAb-G at 37° C. but had 3 “white” attributes (i.e., values in white boxes in FIG. 17, which indicate large improvement in precipitation kinetics) at 25° C. While 1% Poly1 did not improve precipitation (FIG. 18A-B), 0.15% Poly1 did by a statistically significant amount compared to no additive during the first 48 hours of the experiment when added at 0.15% (FIG. 19A). Higher concentration of polymer likely leads to steric crowding with the mAb, overwhelming any beneficial effects. Additionally, both Poly22 and Poly11 are less hydrophobic than Poly1, which may contribute to the difference in their effect on mAb-G precipitation compared to Poly1. From these results, 0.15% Poly1 was selected for continued evaluation.

### B. Polymer Viscosity

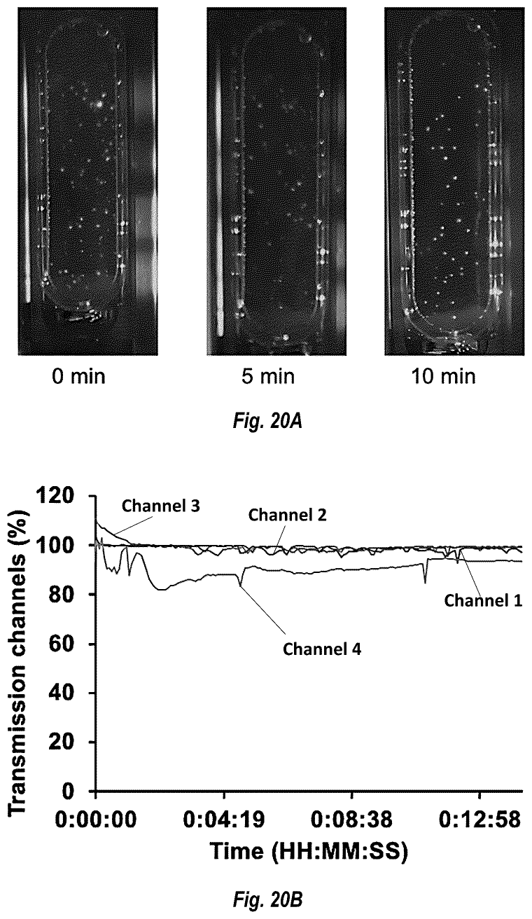

It was assumed that the protein concentration in vivo upon subcutaneous (s.c.) injection would gradually decrease as mAb diffuses away from the injection site. It was expected that addition of the polymer might prevent injection site reactions elicited by protein precipitates provided the protein concentration falls below the solubility limit before the time to precipitation. To evaluate the precipitation more fully in vitro and the effect of changing mAb-G concentration as a result of diffusion, an instrument developed for the in vitro evaluation of s.c. mAb pharmacokinetics (PK), the SCISSOR system, was used (as described in Example 1). This system employs a sample cartridge containing hyaluronic acid (HA) in a buffer with dialysis membrane tuned to mimic mAb s.c. PK in humans (Bown et al., 2018a; Kinnunen et al., 2015). The pH and temperature of the apparatus is also monitored and controlled. It is capable of measuring the inline percent transmittance of the cartridge throughout the experiment and aliquots from the bulk reservoir are automatically removed for offline analysis. Interestingly, mAb-G precipitation was observed immediately upon injection during initial experiments as opposed to previous in vitro experiments in which the mAb precipitation was observed on the order of hours. This may be due to steric crowding and increased viscosity of the HA matrix employed. The injection volume was calibrated by injections of 50 mg/mL mAb-G (FIG. 20A-B), which we observed from in vivo experiments, is the highest concentration that does not elicit injection site inflammation with mAb-G in vivo (data not shown). Future experiments to investigate the effects of cartridge matrix on the precipitation may improve this model for evaluation of precipitation, as cartridge conditions were developed to correlate with mAb PK in humans and do not necessarily reflect the physiological composition of the s.c. space.

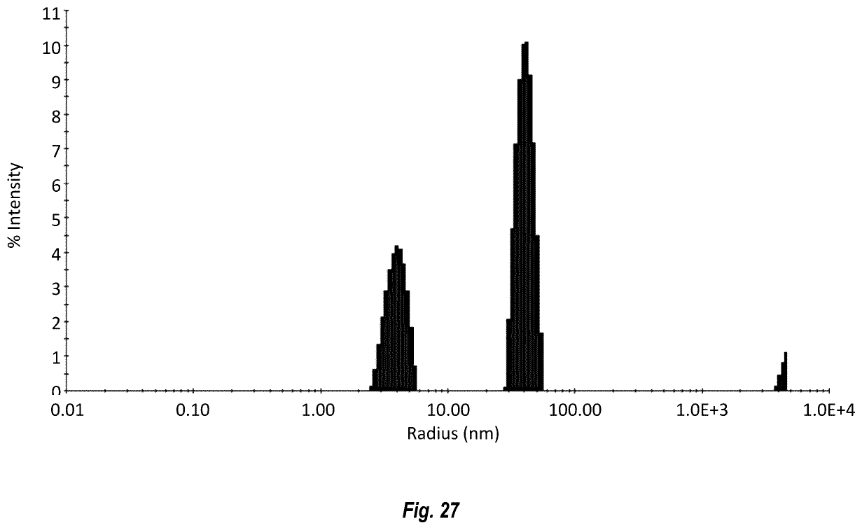

First, the precipitation and PK of mAb-G without any additive was measured by in-line transmittance, offline protein concentration quantitation via Bradford assay, and recording images of the cartridge through the cell window. Upon injection of mAb-G into the cartridge, the protein precipitated as illustrated by the decrease in transmission from the bottom most channel (Channel 4) to 60% within 5 minutes (FIG. 19C) and images of the cartridge (FIG. 19D). The extent of precipitation was decreased when mAb-G was formulated with 0.15% Poly11 as seen in the cartridge images (FIG. 19D). The transmittance data from channel 4, which increases to around 80% within 5 minutes (FIG. 19C), corroborates the reduced extent of precipitation. The release of mAb-G from the cartridge as measured by Bradford assay showed similar initial release rates with and without Poly1 (FIG. 19F), with slightly less total release of mAb-G when Poly1 was present. Note, the total amount of released mAb-G was only statistically significantly different after about 270 min (p<0.05). This difference may be due to protein-polymer interactions, which can alter the diffusion of mAb-G and its interaction with the HA matrix. Additionally, the PK of the polymer alone was using a polymer fluorescently labeled with FITC. The polymer cleared faster than mAb-G (FIG. 19G), as expected due to its lower molecular weight (10 kDa). However, the rate of release also suggests that interactions occur between the slightly positively charged polymer and negatively charged HA matrix. Higher error in the replicates may result from heterogeneity in the micelles formed from self-assembly of the random heteropolymer due to its amphiphilicity, as illustrated in DLS (FIG. 27).

### Example 4. Properties of the Poly1 Polymer

### A. Poly1 as Surfactant

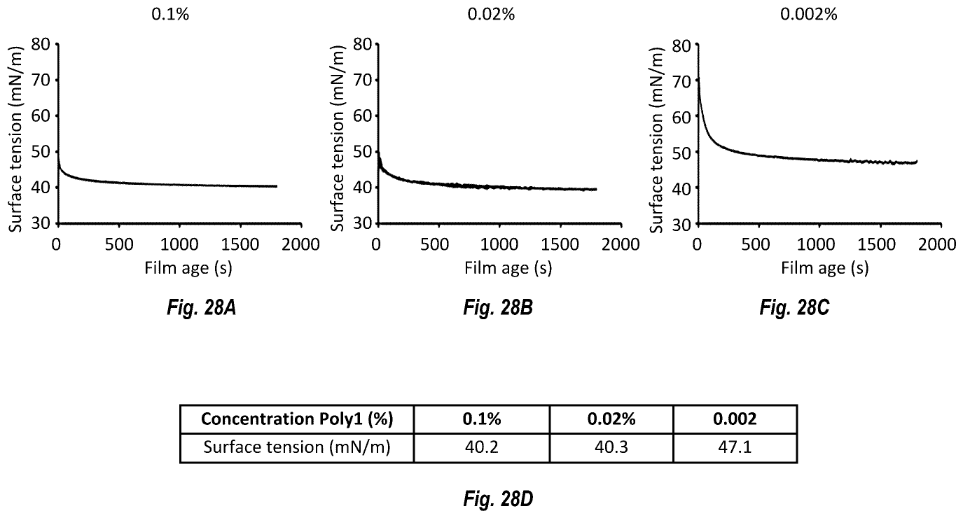

The mechanism by which the polymer improves colloidal stability was also investigated. Because the polymers include a mixture of hydrophobic and hydrophilic monomers, it was hypothesized that the polymers have the potential to act as surfactants and form micelles. Surface tension (ST) measured (as described in Example 1 above) by pendant drop tensiometer agreed with this hypothesis. The critical micelle concentration (CMC) of Poly1 was found to be between 0.002 and 0.02% by measuring the ST at several polymer concentrations (FIGS. 28A-D). Poly1 above its CMC decreased the ST of water from 72 to 40.3 mN/m, which was similar to the ST decrease observed using polysorbate 20 (PS20) above its CMC (Mittal, 1972) (Table 4). Interestingly, Poly1 altered precipitation kinetics in the plate-based absorbance assay, whereas PS20 did not (FIGS. 16A-D). Thus, we hypothesized that an additional mechanism was at play in this system.

TABLE 4
Surface Tension of Water with Surfactants (n = 3)

| Sample | Surface tension (mN/m) |
|---|---|
| Water | 72 |
| Poly1 (0.02%) | 40.3 |
| PS20 (0.05%) | 36.3 |

### B. Microscale Thermophoresis (MST)

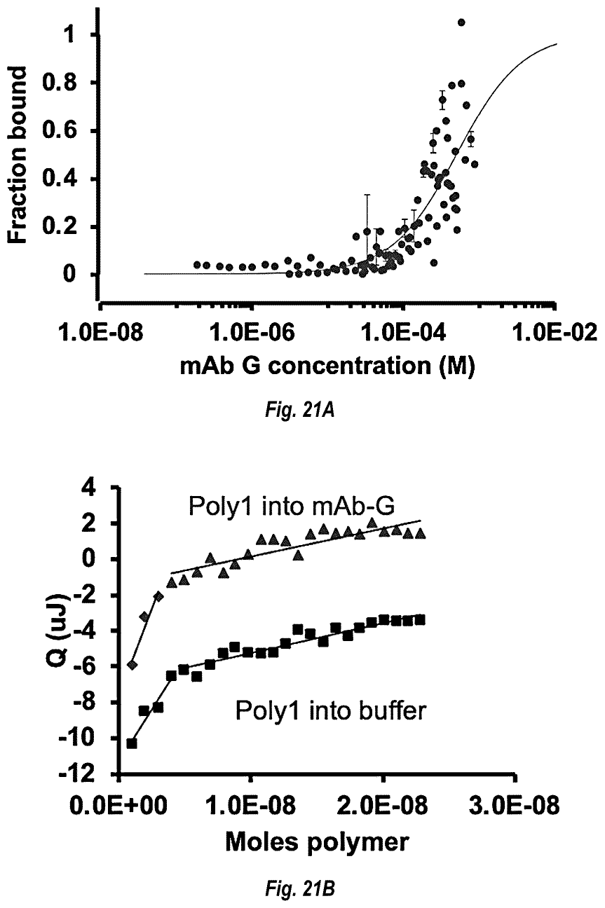

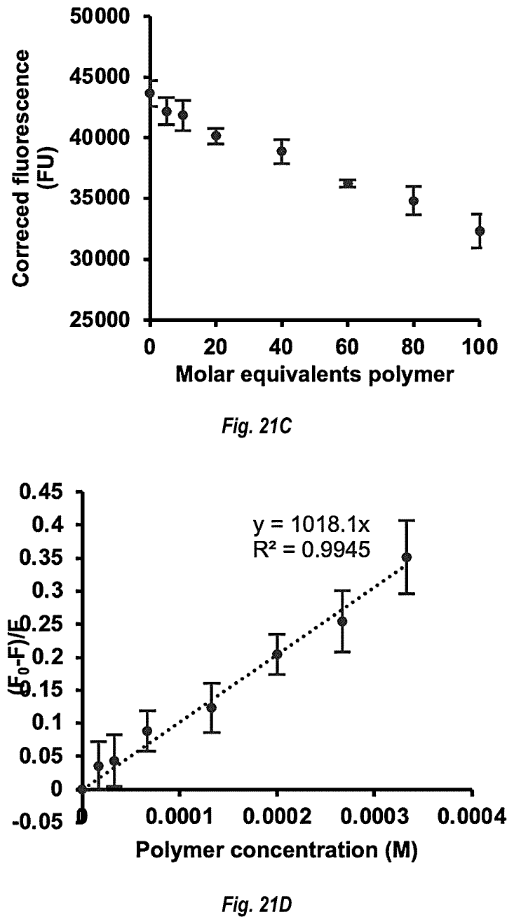

Poly1 was evaluated for interaction with mAb-G through intermolecular interactions including electrostatic interactions, hydrogen-bonding, and hydrophobic/Van der Waals interactions. To evaluate the binding of Poly1 to protein several methods were employed. First, microscale thermophoresis (MST; as described in Example 1 above) was initially used to evaluate the binding of the Poly1 with mAb-G because of its sensitivity to changes in interactions and small volume requirements (FIG. 21A) (Jerabek-Willemsen et al., 2011). Constant Poly1 concentration with increasing amounts of protein were used and a partial binding curve with an estimated dissociation constant (K_{d})>0.5 mM was calculated. A full binding curve with true quantification of the binding was not achieved for protein solubility and viscosity above 1 mM.

### C. Isothermal Titration Calorimetry (ITC)

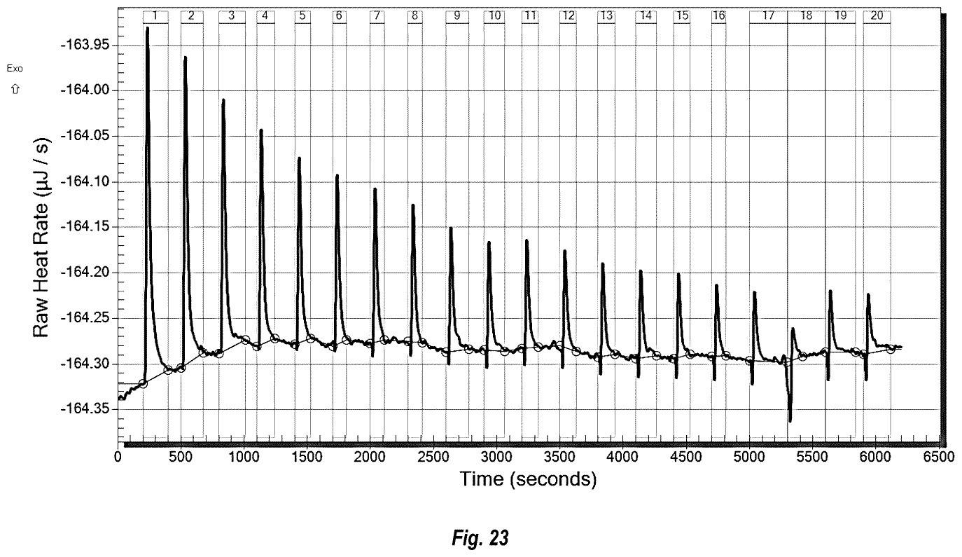

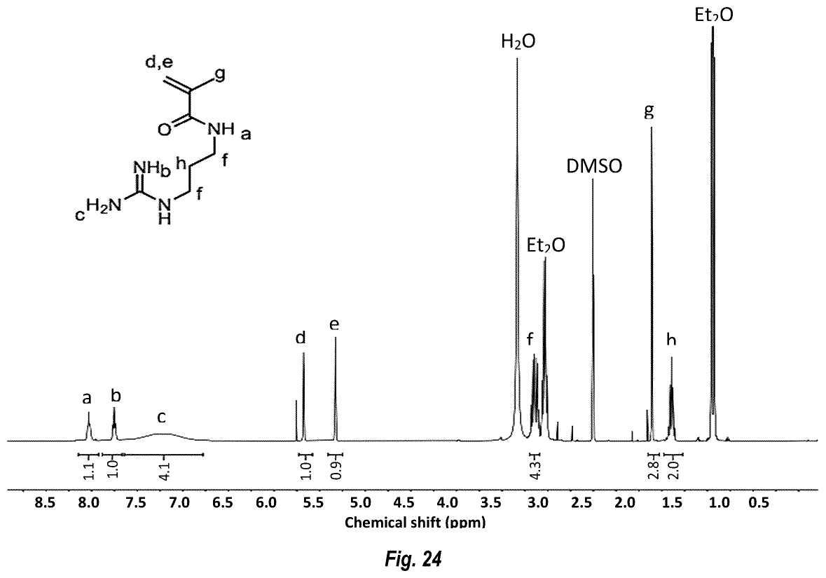

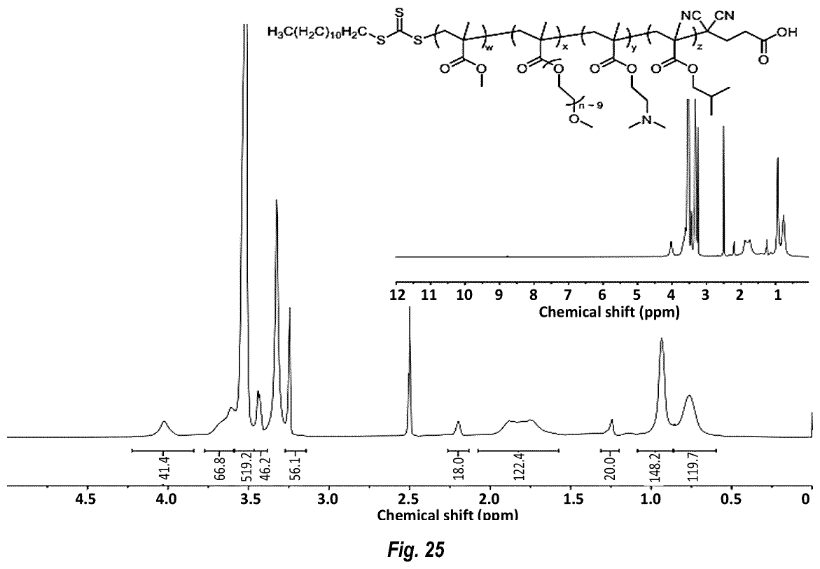

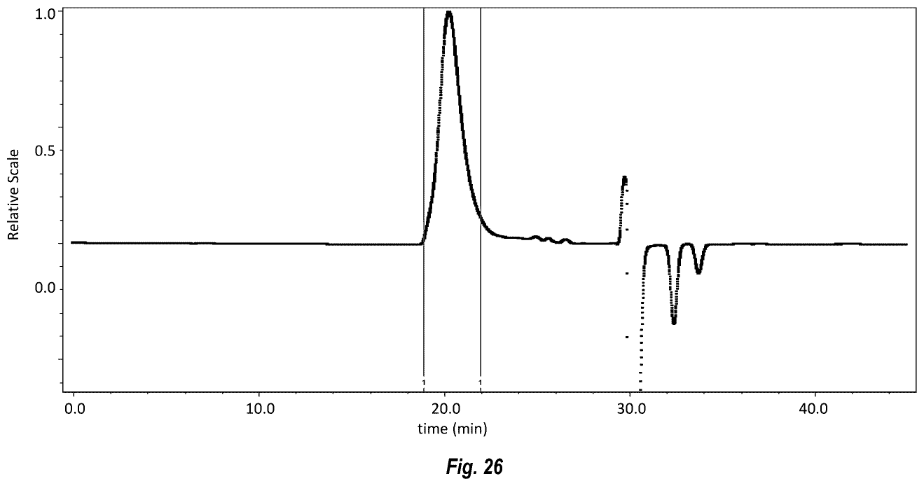

Next, isothermal titration calorimetry (ITC; as described in Example 1 above) was also used to evaluate the enthalpy of the binding interaction (FIG. 21B). The viscosity above 1 mM for mAb-G and 100 μM for Poly1 prevented generation of a full ITC binding curve (FIG. 23). However, the millimolar concentrations used indicated the order of magnitude of interactions. For redundancy, the enthalpy of binding was determined by measuring the enthalpy of demicellization and the critical micelle concentration (CMC) (Bam et al., 1998). The total heat of demicellization, polymer dilution, and protein-polymer binding were determined from the heat released during titration of polymer into a buffer solution and into a mAb-G solution. The CMC estimated by this technique was 0.01 mM (0.01%), which is consistent with the results from ST measurements (Table 4). The selected concentration, which alters precipitation kinetics of mAB-G, exceeds the CMC to ensure sufficient polymer concentration in the bulk versus the surface. The enthalpy of binding of the polymer with mAb-G, as calculated from the change in the initial slope of titrating polymer into mAb-G, approximately −0.18 kcal/mol, indicates a weak interaction between the two species. The endothermic heat of binding indicates entropically-driven binding likely due to hydrophobic interactions and release of bound “structured” water (Ladbury & Chowdhry, 1996). As expected, intrinsic fluorescence decreased with increasing amounts of polymer. The molar equivalents of polymer to achieve this decrease on the order of 10-100 further indicated the weak nature of this interaction. To ensure that the decrease in fluorescence resulted from binding, and not an artifact from the inner-filter effect or collisional quenching (van de Weert & Stella, 2011a), control experiments were performed as detailed in Example 1. Assuming 1:1 binding, the association constant (Kₐ) was calculated from the slope of the Stern-Volmer plot (FIG. 21D) and the corresponding K_{d} was estimated to be ˜0.9 mM, the same order of magnitude as the MST results.

### D. Intrinsic Fluorescence of Poly1

Finally, intrinsic fluorescence quenching (as described in Example 1 above) was used to qualitatively corroborate the findings of the other two techniques (FIG. 21C-D). The intrinsic tryptophan fluorescence was measured in the presence of increasing concentrations of Poly1 (FIG. 21C). As expected, intrinsic fluorescence decreased with increasing amounts of Poly1. The molar equivalents of Poly1 to achieve this decrease were on the order of 10-100, again indicating the weak nature of this interaction. To ensure that the decrease in fluorescence was due to binding and not artifacts from the inner-filter effect or collisional quenching as described in the literature by van de Weert and Stella, control experiments were included as detailed in the Methods and Materials (van de Weert & Stella, 2011b). Assuming 1:1 binding, the association constant (Kₐ) was calculated from the slope of the Stern-Volmer plot (FIG. 21D) and the corresponding K_{d} was estimated to be ˜0.9 mM, the same order of magnitude as the MST results.

E. Summary of Poly1 and mAB-G Interactions

From the biophysical studies, inclusion of Poly1 likely altered mAb-G precipitation through binding equilibrium. Similar studies investigating viscosity reduction via reversible PEGylation of high concentration mAb formulations also demonstrated that PEG binding with many proteins within a short time period disrupted mAb self-interaction (Gong et al., 2019). PEG binding with many proteins within a short time period disrupted mAb self-interaction (Gong et al., 2019). Binding experiments with surface plasmon resonance (SPR) required high concentrations (100 mM) of mPEG-phenylglyoxal to generate an association/dissociation curve, similar to the need to increase concentration to measure binding in this report. For the random heteropolymers described herein, the molecular weight, strength of binding, the number of binding sites, and additional mechanisms like micellization also contribute to the mechanism of precipitation inhibition.

### F. Cytotoxicity of Poly1

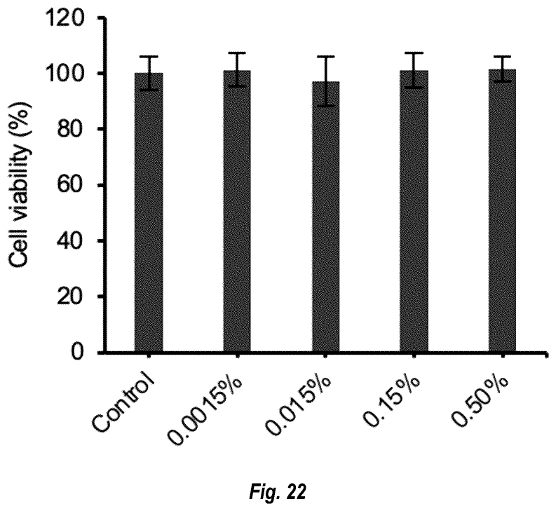

The polymer excipients described in the Examples herein may be used, in some cases, in pharmaceutical formulations, such as for pharmaceutical proteins, including antibodies, and subcutaneous (s.c.) formulations, which may require high protein concentrations. Thus, Poly1 toxicity was investigated in vitro. Because Poly1 is a surfactant, the membrane integrity of cells was used as a measure of cytotoxicity with the trypan blue assay. Jurkat cells were used to model toxicity to immune cells and for their experimental ease as a suspension cell line. Addition of Poly1 at all tested concentrations did not significantly alter the cell viability (FIG. 22). Immunogenicity, biocompatibility, and bioaccumulation of polymers are also considerations for the development of new polymer excipients for pharmaceutical formulations. Immune responses to polymers are generally thought to be caused by the flexibility and regular repetitive structures. The polymers described herein contain a relatively random distribution of monomers, giving them an advantage over other polymers. Anti-PEG antibodies have been observed in some products and bioconjugates containing PEG (Kozma et al., 2020; Zhang et al., 2016). However, the length of the PEG side chain for the OEGMA500 monomer (500 Da) is less than the approximately 750 Da required to bind/generate anti-PEG antibodies (Huckaby et al., 2020). Finally, the SCISSOR experiments indicated the polymer will likely clear from the SC tissue into circulation at a similar or slightly faster rate than mAb-G. Additionally, the polymers reported here are approximately 10 kDa, which is smaller than the approximately 30 kDa molecular weight limit for kidney clearance associated with PEG (Bertrand & Leroux, 2012). Thus, the polymers are expected to be clearable.

### DISCUSSION

As described in the Examples herein, a library of random heteropolymers was synthesized and was screened for the ability of the polymers in the library to improve the solubility of a monoclonal antibody. Polymers were synthesized and purified in a high throughput manner. A plate-based absorbance turbidity assay was used to screen the polymers for their effect on the solubility of a monoclonal antibody, mAb-G, which which precipitates at physiological pH and salt. Selected polymers were studied further for their solubilization effects on mAb-G in increasingly relevant conditions (temperature, in the presence of HA as a simulated matrix). Mechanistic studies showed that the polymers acted as surfactants and could weakly interact with mAb-G to alter the kinetics of precipitation. In sum, random heteropolymers are a promising new class of excipients that may improve the safety and the case of formulating poorly soluble proteins at high concentrations, particularly for subcutaneous administration.

The change in precipitation kinetics in the presence of various polymers from the initial solubility screen at 25° C. (FIG. 15) did not fully correspond to results from the continued study at 37° C. (FIGS. 18A-B and 19A-B). For example, Poly22 and Poly11 had later tₒₙₛₑₜ and lower AUC, and Poly22 had lower Aₘₐₓ than Poly1 at 25° C. (FIGS. 15 and 17), but only Poly1 extended the time to precipitation at 37° C. (FIG. 19A-B). Additionally, only 1% Poly11 made precipitation of mAb-G worse, compared to 1% Poly1, Poly22, and Poly7, which were the same as the control (FIG. 17). Of these polymers, Poly11 and Poly22 are the least hydrophobic, with a relatively higher molar percentage of mOEGMA500, with Poly22 incorporating ArgMAm instead of DMAEMA. These results combined with the ITC experiment (FIG. 21B), indicate hydrophobic interactions between polymer and mAb are likely important and comptribute to the structure-property relationship that delineates Poly1 as the best polymer to improve mAb-G colloidal stability. The temperature dependence of the mAb-G precipitation time scale, with the slowest kinetics at about room temperature (25° C.) and faster kinetics occurring at higher (37° C.) temperatures, again illustrates the importance of hydrophobic interactions. Increased structural flexibility exposing more hydrophobic residuals coupled with increased desolvation producing greater entropy gains (Chen et al., 2003) and increasing strength of hydrophobic interactions (Baldwin, 1986) at higher temperatures may underlie this change. Additionally, differences in the rate of diffusion and/or solubility of aggregates may contribute to this temperature dependence (Wang & Roberts, 2018). Overall, the kinetics of the precipitation process and how the random heteropolymers affect the early stages/seeding of precipitation seem to be important in this system.

The polymer excipients described in the Examples herein may be used, in some cases, in pharmaceutical formulations, such as for pharmaceutical proteins, including antibodies, and subcutaneous (s.c.) formulations, which may require high protein concentrations. Immunogenicity, biocompatibility, and bioaccumulation of polymers are each considerations for the development of new polymer excipients for pharmaceutical formulations. Immune responses to polymers are generally thought to be caused by the flexibility and regular repetitive structures. The polymers described herein contain a relatively random distribution of monomers, giving them an advantage over other polymers.

Another aspect of pharmaceutical formulation development, for example for s.c. antibody formulations and the like, is ensuring product quality during the manufacture, storage, and administration of these therapeutics. Precipitation observed during any of these stages, including upon injection can limit the development of mAbs, particularly if they are intended for s.c. administration or must be formulated at high concentration. For the mAb described herein, the relatively high salt and pH under physiologically relevant conditions led to increased noncovalent intermolecular interactions resulting in precipitation (Pindrus et al., 2015). The observable difference in precipitation kinetics of mAb-G in simulated s.c. matrices indicate the random heteropolymer excipients may enable delivery of poorly soluble mAbs by s.c. injection.

### REFERENCES

Alconcel, S. N. S., Baas, A. S., & Maynard, H. D. (2011). FDA-approved poly(ethylene glycol)-protein conjugate drugs. Polymer Chemistry, 2 (7), 1442-1448. doi.org/10.1039/C1PY00034A
Arlotta, K. J., & Owen, S. C. (2019). Antibody and antibody derivatives as cancer therapeutics. Wiley Interdisciplinary Reviews. Nanomedicine and Nanobiotechnology, 11 (5), e1556. doi.org/10.1002/wnan.1556
Baldwin, R. L. (1986). Temperature dependence of the hydrophobic interaction in protein folding. Proceedings of the National Academy of Sciences of the United States of America, 83 (21), 8069-8072. https://doi.org/10.1073/pnas.83.21.8069 Bam, N. B., Cleland, J. L., Yang, J., Manning, M. C., Carpenter, J. F., Kelley, R. F., & Randolph∥, T. W. (1998). Tween protects recombinant human growth hormone against agitation-induced damage via hydrophobic interactions. Journal of Pharmaceutical Sciences, 87 (12), 1554-1559. doi.org/10.1021/js980175ν
Bertrand, N., & Leroux, J.-C. (2012). The journey of a drug-carrier in the body: An anatomo-physiological perspective. Journal of Controlled Release, 161 (2), 152-163. doi.org/10.1016/j.jconrel.2011.09.098
Bown, H. K., Bonn, C., Yohe, S., Yadav, D. B., Patapoff, T. W., Daugherty, A., & Mrsny, R. J. (2018). In vitro model for predicting bioavailability of subcutaneously injected monoclonal antibodies. Journal of Controlled Release, 273, 13-20. doi.org/10.1016/j.jconrel.2018.01.015
Chen, W.-Y., Huang, H.-M., Lin, C.-C., Lin, F.-Y., & Chan, Y.-C. (2003). Effect of Temperature on Hydrophobic Interaction between Proteins and Hydrophobic Adsorbents: Studies by Isothermal Titration calorimetry and the van′t Hoff Equation. Langmuir, 19 (22), 9395-9403. doi.org/10.1021/la0347830
Crommelin, D. J. A., Mastrobattista, E., Hawe, A., Hoogendoorn, K. H., & Jiskoot, W. (2020). Shifting Paradigms Revisited: Biotechnology and the Pharmaceutical Sciences. Journal of Pharmaceutical Sciences, 109 (1), 30-43. doi.org/10.1016/j.xphs.2019.08.010
Dubacheva, G. V., Curk, T., Mognetti, B. M., Auzély-Velty, R., Frenkel, D., & Richter, R. P. (2014). Superselective targeting using multivalent polymers. Journal of the American Chemical Society, 136 (5), 1722-1725. doi.org/10.1021/ja411138s
Funhoff, A. M., van Nostrum, C. F., Lok, M. C., Fretz, M. M., Crommelin, D. J. A., & Hennink, W. E. (2004). Poly(3-guanidinopropyl methacrylate): A Novel Cationic Polymer for Gene Delivery. Bioconjugate Chemistry, 15 (6), 1212-1220. doi.org/10.1021/bc049864q
Garidel, P., Kuhn, A. B., Schäfer, L. V., Karow-Zwick, A. R., & Blech, M. (2017). High-concentration protein formulations: How high is high? European Journal of Pharmaceutics and Biopharmaceutics: Official Journal of Arbeitsgemeinschaft Fur Pharmazeutische Verfahrenstechnik e. V, 119, 353-360. doi.org/10.1016/j.cjpb.2017.06.029
Gong, Y., Soleymani Abyanch, H., Drossis, N., Niederquell, A., Kuentz, M., Leroux, J.-C., de Haan, H. W., & Gauthier, M. A. (2019). Ultra-sub-stoichiometric “Dynamic” Bioconjugation Reduces Viscosity by Disrupting Immunoglobulin Oligomerization. Biomacromolecules, 20 (9), 3557-3565. doi.org/10.1021/acs.biomac.9b00867
Gormley, A. J., Ycow, J., Ng, G., Conway, Ó., Boyer, C., & Chapman, R. (2018). An Oxygen-Tolerant PET-RAFT Polymerization for Screening Structure-Activity Relationships. Angewandte Chemie (International Ed. in English), 57 (6), 1557-1562. doi.org/10.1002/anie.201711044
Grubbs, R. B., & Grubbs, R. H. (2017). 50th Anniversary Perspective: Living Polymerization-Emphasizing the Molecule in Macromolecules. Macromolecules, 50 (18), 6979-6997. doi.org/10.1021/acs.macromol.7b01440
Huckaby, J. T., Jacobs, T. M., Li, Z., Perna, R. J., Wang, A., Nicely, N. I., & Lai, S. K. (2020). Structure of an anti-PEG antibody reveals an open ring that captures highly flexible PEG polymers. Communications Chemistry, 3 (1), 1-8. doi.org/10.1038/s42004-020-00369-y
Jerabek-Willemsen, M., Wienken, C. J., Braun, D., Baaske, P., & Duhr, S. (2011). Molecular Interaction Studies Using Microscale Thermophoresis. Assay and Drug Development Technologies, 9 (4), 342-353. doi.org/10.1089/adt.2011.0380
Kamerzell, T. J., Pace, A. L., Li, M., Danilenko, D. M., McDowell, M., Gokarn, Y. R., & Wang, Y. J. (2013). Polar solvents decrease the viscosity of high concentration IgG1 solutions through hydrophobic solvation and interaction: Formulation and biocompatibility considerations. Journal of Pharmaceutical Sciences, 102 (4), 1182-1193. doi.org/10.1002/jps.23453
Kinnunen, H. M., Sharma, V., Contreras-Rojas, L. R., Yu, Y., Alleman, C., Sreedhara, A., Fischer, S., Khawli, L., Yohe, S. T., Bumbaca, D., Patapoff, T. W., Daugherty, A. L., & Mrsny, R. J. (2015). A novel in vitro method to model the fate of subcutaneously administered biopharmaceuticals and associated formulation components. Journal of Controlled Release, 214, 94-102. doi.org/10.1016/j.jconrel.2015.07.016
Kozma, G. T., Shimizu, T., Ishida, T., & Szebeni, J. (2020). Anti-PEG antibodies: Properties, formation, testing and role in adverse immune reactions to PEGylated nano-biopharmaceuticals. Advanced Drug Delivery Reviews, 154-155, 163-175. doi.org/10.1016/j.addr.2020.07.024
Laaser, J. E., Jiang, Y., Sprouse, D., Reineke, T. M., & Lodge, T. P. (2015). PH- and Ionic-Strength-Induced Contraction of Polybasic Micelles in Buffered Aqueous Solutions. Macromolecules, 48 (8), 2677-2685. doi.org/10.1021/acs.macromol.5b00360
Ladbury, J. E., & Chowdhry, B. Z. (1996). Sensing the heat: The application of isothermal titration calorimetry to thermodynamic studies of biomolecular interactions. Chemistry & Biology, 3 (10), 791-801. doi.org/10.1016/S1074-5521 (96) 90063-0
Lee, H., Son, S. H., Sharma, R., & Won, Y.-Y. (2011). A discussion of the pH-dependent protonation behaviors of poly(2-(dimethylamino)ethyl methacrylate) (PDMAEMA) and poly(ethylenimine-ran-2-ethyl-2-oxazoline) (P (EI-r-EOz)). The Journal of Physical Chemistry. B, 115 (5), 844-860. doi.org/10.1021/jp109151s
Lodge, T. P., & Muthukumar, M. (1996). Physical chemistry of polymers: Entropy, interactions, and dynamics. Journal of Physical Chemistry, 100 (31), 13275-13292. doi.org/10.1021/jp960244z
Lu, R.-M., Hwang, Y.-C., Liu, I.-J., Lee, C.-C., Tsai, H.-Z., Li, H.-J., & Wu, H.-C. (2020). Development of therapeutic antibodies for the treatment of diseases. Journal of Biomedical Science, 27 (1), 1. doi.org/10.1186/s12929-019-0592-z
Mann, J. L., Maikawa, C. L., Smith, A. A. A., Grosskopf, A. K., Baker, S. W., Roth, G. A., Meis, C. M., Gale, E. C., Liong, C. S., Correa, S., Chan, D., Stapleton, L. M., Yu, A. C., Muir, B., Howard, S., Postma, A., & Appel, E. A. (2020). An ultrafast insulin formulation enabled by high-throughput screening of engineered polymeric excipients. Science Translational Medicine, 12 (550), eaba6676. doi.org/10.1126/scitranslmed.aba6676
Matyjaszewski, K., & Xia, J. (2001). Atom transfer radical polymerization. Chemical Reviews, 101 (9), 2921-2990. doi.org/10.1021/cr940534g
Mittal, K. L. (1972). Determination of CMC of polysorbate 20 in aqueous solution by surface tension method. Journal of Pharmaceutical Sciences, 61 (8), 1334-1335. doi.org/10.1002/jps.2600610842
Ng, G., Ycow, J., Chapman, R., Isahak, N., Wolvetang, E., Cooper-White, J. J., & Boyer, C. (2018). Pushing the Limits of High Throughput PET-RAFT Polymerization. Macromolecules, 51 (19), 7600-7607. doi.org/10.1021/acs.macromol.8b01600
Oliver, S., Zhao, L., Gormley, A. J., Chapman, R., & Boyer, C. (2019). Living in the Fast Lane-High Throughput Controlled/Living Radical Polymerization. Macromolecules, 52 (1), 3-23. doi.org/10.1021/acs.macromol.8b01864
Panganiban, B., Qiao, B., Jiang, T., DelRe, C., Obadia, M. M., Nguyen, T. D., Smith, A. A. A., Hall, A., Sit, I., Crosby, M. G., Dennis, P. B., Drockenmuller, E., Olvera de la Cruz, M., & Xu, T. (2018). Random heteropolymers preserve protein function in foreign environments. Science (New York, N.Y.), 359 (6381), 1239-1243. doi.org/10.1126/science.aao0335
Perrier, S. (2017). 50th Anniversary Perspective: RAFT Polymerization-A User Guide. Macromolecules, 50 (19), 7433-7447. doi.org/10.1021/acs.macromol.7b00767
Pindrus, M., Shire, S. J., Kelley, R. F., Demeule, B., Wong, R., Xu, Y., & Yadav, S. (2015). Solubility Challenges in High Concentration Monoclonal Antibody Formulations: Relationship with Amino Acid Sequence and Intermolecular Interactions. Molecular Pharmaceutics, 12 (11), 3896-3907. doi.org/10.1021/acs.molpharmaceut.5b00336
Pivot, X., Gligorov, J., Müller, V., Curigliano, G., Knoop, A., Verma, S., Jenkins, V., Scotto, N., Osborne, S., Fallowfield, L., Fallowfield, L., Jenkins, V., Kilkerr, J., Langridge, C., Monson, K., Jakobsen, E. H., Nielsen, M. H., Linnet, S., Knoop, A., . . . . Verma, S. (2014). Patients' preferences for subcutaneous trastuzumab versus conventional intravenous infusion for the adjuvant treatment of HER2-positive early breast cancer: Final analysis of 488 patients in the international, randomized, two-cohort PrefHer study. Annals of Oncology, 25 (10), 1979-1987. doi.org/10.1093/annonc/mdu364
Shire, S. J., Shahrokh, Z., & Liu, J. (2004). Challenges in the development of high protein concentration formulations. Journal of Pharmaceutical Sciences, 93 (6), 1390-1402. doi.org/10.1002/jps.20079
Shukla, D., & Trout, B. L. (2010). Interaction of arginine with proteins and the mechanism by which it inhibits aggregation. The Journal of Physical Chemistry. B, 114 (42), 13426-13438. doi.org/10.1021/jp108399g
Stoner, K. L., Harder, H., Fallowfield, L. J., & Jenkins, V. A. (2014). Intravenous versus Subcutaneous Drug Administration. Which Do Patients Prefer? A Systematic Review. The Patient. doi.org/10.1007/s40271-014-0075-y
Sudrik, C., Cloutier, T., Pham, P., Samra, H. S., & Trout, B. L. (2017). Preferential interactions of trehalose, L-arginine.HCl and sodium chloride with therapeutically relevant IgG1 monoclonal antibodies. MAbs, 9 (7), 1155-1168. doi.org/10.1080/19420862.2017.1358328
Thakral, S., Thakral, N. K., & Majumdar, D. K. (2013). Eudragit®: A technology evaluation. Expert Opinion on Drug Delivery, 10 (1), 131-149. doi.org/10.1517/17425247.2013.736962
Ting, J. M., Tale, S., Purchel, A. A., Jones, S. D., Widanapathirana, L., Tolstyka, Z. P., Guo, L., Guillaudeu, S. J., Bates, F. S., & Reineke, T. M. (2016). High-Throughput Excipient Discovery Enables Oral Delivery of Poorly Soluble Pharmaceuticals. ACS Central Science, 2 (10), 748-755. doi.org/10.1021/acscentsci.6b00268
van de Weert, M., & Stella, L. (2011a). Fluorescence quenching and ligand binding: A critical discussion of a popular methodology. J. Mol. Struct., 998, 144-150. doi.org/10.1016/j.molstruc.2011.05.023
Viola, M., Sequeira, J., Seica, R., Veiga, F., Serra, J., Sanros, A. C., & Ribeiro, A. J. (2018). Subcutaneous delivery of monoclonal antibodies: How do we get there?-PubMed. Journal of Controlled Release: Official Journal of the Controlled Release Society, 286, 301-314. doi.org/doi: 10.1016/j.jconrel.2018.08.001
Wang, W., & Roberts, C. J. (2018). Protein aggregation-Mechanisms, detection, and control. International Journal of Pharmaceutics, 550 (1), 251-268. doi.org/10.1016/j.ijpharm.2018.08.043
Zhang, P., Sun, F., Liu, S., & Jiang, S. (2016). Anti-PEG antibodies in the clinic: Current issues and beyond PEGylation. Journal of Controlled Release: Official Journal of the Controlled Release Society, 244 (Pt B), 184-193. doi.org/10.1016/j.jconrel.2016.06.040
Zumbro, E., & Alexander-Katz, A. (2020). Influence of Binding Site Affinity Patterns on Binding of Multivalent Polymers. ACS Omega, 5 (19), 10774-10781. doi.org/10.1021/acsomega.0c00334

What is claimed is:

1. A random polymer library comprising a mixture of polymers, wherein the library comprises polymers comprising at least three monomers chosen from:
a. methyl methacrylate (MMA),
b. oligo(ethylene glycol) methyl ether methacrylate (OEGMA),
c. isobutyl methacrylate (IBMA) or butyl methacrylate (BMA), and
d. a compound of the Formula I, 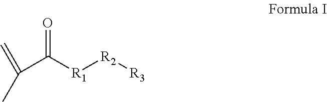
wherein R₁ is O or NH, R₂ is methyl, ethyl, propyl, or butyl, and R₃ is NH₂ or N(CH₃)₂ or a guanidinium group, wherein the library comprises polymers of from 3 to 20 kDa.

2. The random polymer library of claim 1, wherein the library comprises polymers comprising a monomer of the Formula I, wherein the monomer is dimethyl amino methacrylate (DMAEMA), 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide.

3. The random polymer library of claim 1 or 2, wherein at least 50%, at least 60%, at least 70%, at least 80%, or at least 90% of the polymers in the library comprise at least three monomers, or wherein the random polymer library consists essentially of polymers comprising at least three monomers.

4. The random polymer library of claim 1 or 2, wherein at least 50%, at least 60%, at least 70%, at least 80%, or at least 90% of the polymers in the library comprise at least four monomers, or wherein the random polymer library consists essentially of polymers comprising at least four monomers.

5. A random polymer library comprising a mixture of polymers, each polymer comprising a mixture of at least three of monomers: methyl methacrylate (MMA), oligo(ethylene glycol) methyl ether methacrylate (OEGMA), isobutyl methacrylate (IBMA), and dimethyl amino methacrylate (DMAEMA), wherein the library comprises polymers of from 3 to 20 kDa.

6. A random polymer library comprising a mixture of polymers, each polymer comprising a mixture of at least three of monomers: methyl methacrylate (MMA), oligo(ethylene glycol) methyl ether methacrylate (OEGMA), isobutyl methacrylate (IBMA), and N-(3-methacrylamidopropyl) guanidinium chloride (ArgMAm), wherein the library comprises polymers of from 3 to 20 kDa.

7. The random polymer library of any one of claims 1-6, wherein the library comprises polymers of from 3 to 15 kDa, from 5 to 20 kDa, from 5 to 15 kDa, from 5 to 10 kDa, from 10 to 20 kDa, from 7 to 10 kDa, or from 10 to 15 kDa.

8. The random polymer library of any one of claims 1-7, wherein the library comprises OEGMA with a molecular weight of from 300 to 1500 g/mol.

9. The random polymer library of claim 8, wherein the OEGMA has a molecular weight of 300, 500, 750, 950, 1000, 1200, or 1500 g/mol.

10. The random polymer library of claim 8, wherein the OEGMA has a molecular weight of 500 g/mol (i.e., is OEGMA 500).

11. The random polymer library of any one of claim 1-4 or 7-10, wherein library comprises polymers comprising three monomers.

12. The random polymer library of claim 11, wherein at least 50%, at least 60%, at least 70%, at least 80%, or at least 90% of the polymers in the library comprise three monomers, or wherein the random polymer library consists essentially of polymers comprising three monomers.

13. The random polymer library of claim 11 or 12, wherein the three monomers are MMA, OEGMA, and either IBMA or BMA.

14. The random polymer library of claim 13, wherein the three monomers are MMA, OEGMA, and IBMA.

15. The random polymer library of claim 13, wherein the MMA, OEGMA, and IBMA or BMA are present in a ratio of: (a) 5:3:2, or (b) 3:5:2, or (c) 5:4:1.

16. The random polymer library of any one of claims 1-10, wherein the library comprises polymers comprising four monomers.

17. The random polymer library of claim 16, wherein at least 50%, at least 60%, at least 70%, at least 80%, or at least 90% of the polymers in the library comprise three monomers, or wherein the random polymer library consists essentially of polymers comprising three monomers.

18. The random polymer library of claim 16 or 17, wherein the four monomers are MMA, OEGMA, IBMA, and DMAEMA.

19. The random polymer library of claim 18, wherein the polymer library comprises polymers comprising 20-50% MMA, 20-50% OEGMA, 5-25% IBMA, and 5-25% DMAEMA by total weight of polymer.

20. The random polymer library of claim 18, wherein the polymer library comprises polymers comprising MMA, OEGMA, IBMA, and DMAEMA in a molar ratio of: (a) 5:2.5:2:0.5, (b) 2:5:2:1, (c) 5:2.5:1:1.5, (d) 4:4:1:1; (e) 3:4:1:2; (f) 2:4:1:3; and/or (g) 2:3:1:4.

21. The random polymer library of claim 18, wherein the MMA, OEGMA, IBMA, and DMAEMA are in a molar ratio of 5:2.5:2:0.5, wherein the OEGMA has a molecular weight of 500 g/mol (i.e., is OEGMA 500), wherein the library comprises polymers with a molecular weights of 3-15 kDa.

22. The random polymer library of claim 16 or 17, wherein the four monomers are MMA, OEGMA, IBMA, and ArgMAm.

23. The random polymer library of claim 22, wherein the polymer library comprises polymers comprising 20-50% MMA, 20-50% OEGMA, 5-25% IBMA, and 5-25% ArgMAm by total weight of polymer.

24. The random polymer library of claim 22, wherein the polymer library comprises polymers comprising MMA, OEGMA, IBMA, and ArgMAm in a molar ratio of: (a) 5:2.5:2:0.5, (b) 2:5:2:1, (c) 5:2:1:2, (d) 4:4:1:1; (e) 3:4:1:2; (f) 2:4:1:3; and/or (g) 2:3:1:4.

25. The random polymer library of claim 22, wherein the MMA, OEGMA, IBMA, and ArgMAm are in a molar ratio of 5:2:1:2 or 4:4:1:1, wherein the OEGMA has a molecular weight of 500 g/mol (i.e., is OEGMA 500), wherein the library comprises polymers with a molecular weights of 3-15 kDa.

26. A random polymer library comprising a mixture of polymers, wherein the library comprises polymers comprising at least two monomers chosen from oligo(ethylene glycol) methyl ether methacrylate (OEGMA) and either isobutyl methacrylate (IBMA) or butyl methacrylate (BMA), and/or at least two monomers chosen from oligo(ethylene glycol) methyl ether methacrylate (OEGMA) and N-(3-methacrylamidopropyl) guanidinium chloride (ArgMAm), optionally wherein the OEGMA has a molecular weight of 500 g/mol (i.e., is OEGMA 500), and optionally wherein the library comprises polymers with a molecular weights of 3-15 kDa.

27. A random polymer comprising a mixture of at least three monomers chosen from:
a. methyl methacrylate (MMA),
b. oligo(ethylene glycol) methyl ether methacrylate (OEGMA),
c. isobutyl methacrylate (IBMA) or butyl methacrylate (BMA), and
d. a compound of the Formula I, 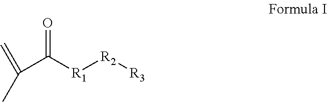
wherein R₁ is O or NH, R₂ is methyl, ethyl, propyl, or butyl, and R₃ is NH₂ or N(CH₃)₂ or a guanidinium group, wherein the polymer is from 3 to 20 kDa.

28. The random polymer of claim 27, wherein the polymer comprises a monomer of the Formula I, 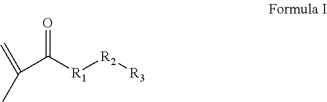
wherein the monomer is dimethyl amino methacrylate (DMAEMA), 2-aminoethyl methacrylate, arginine methacrylate, arginine methacrylamide, N-(3-aminopropyl) methacrylamide, N-(3-methacrylamidopropyl) guanidinium chloride (ArgMAm), or N-3-(dimethylamino) propyl methacrylamide.

29. A random polymer comprising a mixture of at least three of monomers: methyl methacrylate (MMA), oligo(ethylene glycol) methyl ether methacrylate (OEGMA), isobutyl methacrylate (IBMA), and dimethyl amino methacrylate (DMAEMA), wherein the polymer is from 3 to 20 kDa.

30. A random polymer comprising a mixture of at least three of monomers: methyl methacrylate (MMA), oligo(ethylene glycol) methyl ether methacrylate (OEGMA), isobutyl methacrylate (IBMA), and N-(3-methacrylamidopropyl) guanidinium chloride (ArgMAm), wherein the polymer is from 3 to 20 kDa.

31. The random polymer of any one of claims 27-30, wherein the polymer is from 3 to 15 kDa, from 5 to 20 kDa, from 5 to 15 kDa, from 5 to 10 kDa, from 10 to 20 kDa, from 7 to 10 kDa, or from 10 to 15 kDa.

32. The random polymer of any one of claims 27-31, wherein the polymer comprises OEGMA with a molecular weight of from 300 to 1500 g/mol.

33. The random polymer of claim 32, wherein the OEGMA has a molecular weight of 300, 500, 750, 950, 1000, 1200, or 1500 g/mol.

34. The random polymer of claim 33, wherein the OEGMA has a molecular weight of 500 g/mol.

35. The random polymer of any one of claim 27-28 or 31-34, wherein the polymer comprises three monomers.

36. The random polymer of claim 35, wherein the three monomers are MMA, OEGMA, and either IBMA or BMA.

37. The random polymer of claim 36, wherein the three monomers are MMA, OEGMA, and IBMA.

38. The random polymer of claim 37, wherein the MMA, OEGMA, and IBMA or BMA are present in a ratio of: (a) 5:3:2, or (b) 3:5:2, or (c) 5:4:1.

39. The random polymer of any one of claims 27-34, wherein the polymer comprises four monomers.

40. The random polymer of claim 39, wherein the four monomers are MMA, OEGMA, IBMA, and DMAEMA.

41. The random polymer of claim 40, wherein the polymer comprises 20-50% MMA, 20-50% OEGMA, 5-25% IBMA, and 5-25% DMAEMA by total weight of polymer.

42. The random polymer of claim 40, wherein the polymer comprises MMA, OEGMA, IBMA, and DMAEMA in a molar ratio of: (a) 5:2.5:2:0.5, (b) 2:5:2:1, (c) 5:2.5:1:1.5, (d) 4:4:1:1; (e) 3:4:1:2; (f) 2:4:1:3; or (g) 2:3:1:4.

43. The random polymer of claim 40, wherein the MMA, OEGMA, IBMA, and DMAEMA are in a molar ratio of 5:2.5:2:0.5, wherein the OEGMA is OEGMA 500, wherein the polymer has a molecular weight of from 3 to 15 kDa.

44. The random polymer of claim 39, wherein the four monomers are MMA, OEGMA, IBMA, and ArgMAm.

45. The random polymer of claim 44, wherein the polymer comprises 20-50% MMA, 20-50% OEGMA, 5-25% IBMA, and 5-25% ArgMAm by total weight of polymer.

46. The random polymer of claim 44, wherein the polymer comprises MMA, OEGMA, IBMA, and ArgMAm in a molar ratio of: (a) 5:2.5:2:0.5, (b) 2:5:2:1, (c) 5:2.5:1:1.5, (d) 4:4:1:1; (e) 3:4:1:2; (f) 2:4:1:3; or (g) 2:3:1:4.

47. The random polymer of claim 44, wherein the MMA, OEGMA, IBMA, and ArgMAm are in a molar ratio of 5:2:1:2 or 4:4:1:1, wherein the OEGMA is OEGMA 500, wherein the polymer has a molecular weight of from 3 to 15 kDa.

48. A random polymer comprising mixture of at least two monomers chosen from oligo(ethylene glycol) methyl ether methacrylate (OEGMA) and either isobutyl methacrylate (IBMA) or butyl methacrylate (BMA), and/or at least two monomers chosen from oligo(ethylene glycol) methyl ether methacrylate (OEGMA) and N-(3-methacrylamidopropyl) guanidinium chloride (ArgMAm), optionally wherein the OEGMA has a molecular weight of 500 g/mol (i.e., is OEGMA 500), and optionally wherein the polymer has a molecular weight of from 3 to 15 kDa.

49. The random polymer of any one of claims 27-48, wherein the polymer has a molecular weight of from 7 to 10 kDa.

50. A composition comprising a random polymer according to any one of claims 27-49 and at least one protein.

51. The composition of claim 50, wherein the polymer is at a concentration of from 0.01 to 1% w/v, such as 0.01 to 0.1% w/v.

52. The composition of claim 50 or 51, wherein the protein is an antibody.

53. The composition of claim 52, wherein the antibody is an IgG antibody, such as a full-length IgG, a bi-specific IgG, or wherein the antibody is an antigen binding fragment, such as a Fab, Fab′, (Fab′)2, Fv, or scFv.

54. The composition of any one of claims 50-53, wherein the composition further comprises at least one buffer, such as histidine, phosphate, or citrate.

55. The composition of any one of claims 50-54, wherein the composition further comprises at least one surfactant, such as a polysorbate, for example polysorbate 20 or polysorbate 80.

56. The composition of any one of claims 50-55, wherein the antibody is at a concentration of 10 mg/mL to 150 mg/mL, such as 10-100 mg/mL, 20-100 mg/mL, 20-80 mg/mL, 10-50 mg/mL, 10-25 mg/mL, 25-50 mg/mL, 50-80 mg/mL, 50-100 mg/mL, or 70-100 mg/mL.

57. A method of reducing the viscosity and/or inhibiting precipitation of a protein-containing composition, comprising adding a random polymer of any one of claims 27-49 to the composition, optionally wherein the composition comprises the protein and/or excipients of any one of claims 50-56.

58. A method of preparing a random polymer library according to any one of claims 1-26 or a random polymer according to any one of claims 27-49, wherein the method comprises reverse addition/fragmentation chain transfer (RAFT), free radical polymerization (FRP), or atom transfer radical polymerization (ATRP).

59. A method of preparing a random polymer library according to any one of claims 1-26 or a random polymer according to any one of claims 27-49, the method comprising: exposing the monomers to LED light and/or heat in the presence of a zinc tetraphenyl porphyrin catalyst (ZnTPP) and a chain transfer agent.

60. The method of claim 59, wherein the chain transfer agent is 2-cyano-2-propyl benzodithioate, 2-cyano-2-propyldodecyl trithiocarbonate, 4-((((2-carboxyethyl)thio) carbonothioyl)thio)-4-cyanopentanoic acid, 4-cyano-4-(phenylcarbonothioylthio) pentanoic acid, or 4-cyano-4-[(dodecylsulfanylthiocarbonyl) sulfanyl] pentanoic acid (CDTPA).

61. The method of claim 59 or 60, wherein the monomers are exposed to LED light in the presence of the ZnTPP and chain transfer agent for 3 to 18 hours.

62. The method of any one of claims 59-61, wherein polymerization is ended by removing the LED light.

63. The method of any one of claims 58-62, further comprising performing at least one filtration, buffer exchange, or dialysis step to isolate the polymers.

64. A kit comprising the random polymer library of any one of claims 1-26, for use in testing polymers comprised within the library for their effect on precipitation, viscosity, and/or turbidity of a solution comprising a protein, the kit optionally further comprising instructions for use.

65. The kit of claim 64, wherein the library comprises at least 10, at least 20, at least 50, or at least 80 different individual random polymers.

66. A method of identifying a random polymer excipient for a protein solution, comprising exposing the protein solution to a random polymer library of any one of claims 1-26 or the kit of claim 64 or 65 and determining one or more of the viscosity, turbidity, or precipitation of the solution.
